SEQUENCE LISTING (19 sequences)

SEQ ID NO: 1 - protein, 10 aa, Bacteriophage fd-tet
YERKFWHGRH

SEQ ID NO: 2 - protein, 10 aa, Bacteriophage fd-tet
LHKLVRHGRW

SEQ ID NO: 3 - protein, 10 aa, Bacteriophage fd-tet
YLRRHTHVFW

SEQ ID NO: 4 - protein, 10 aa, Bacteriophage fd-tet
KKHSHYWVRY

SEQ ID NO: 5 - protein, 10 aa, Bacteriophage fd-tet
EFGTRHMRHR

SEQ ID NO: 6 - protein, 10 aa, Bacteriophage fd-tet
YRHHAHGGRG

SEQ ID NO: 7 - protein, 10 aa, Bacteriophage fd-tet
RKKWHGWTRW

SEQ ID NO: 8 - protein, 10 aa, Bacteriophage fd-tet
PKWRHGYTRF

SEQ ID NO: 9 - protein, 10 aa, Bacteriophage fd-tet
RHGTVKHARH

SEQ ID NO: 10 - protein, 10 aa, Bacteriophage fd-tet
RRHWHPPVQI

SEQ ID NO: 11 - protein, 10 aa, Bacteriophage fd-tet
EAYKRRWHWP

SEQ ID NO: 12 - protein, 10 aa, Bacteriophage fd-tet
RWPKRHLSGH

SEQ ID NO: 13 - protein, 10 aa, Bacteriophage fd-tet
SRVPFRHYHH

SEQ ID NO: 14 - protein, 10 aa, Bacteriophage fd-tet
RRPEHTKARW

SEQ ID NO: 15 - protein, 10 aa, Bacteriophage fd-tet
WRAFLPRWHA

SEQ ID NO: 16 - protein, 10 aa, Bacteriophage fd-tet
WNRGWRWWMG

SEQ ID NO: 17 - protein, 10 aa, Bacteriophage fd-tet
GFFWKWRIGR

SEQ ID NO: 18 - protein, 10 aa, Bacteriophage fd-tet
HIRWKGHISW

SEQ ID NO: 19 - protein, 5 aa, Bacteriophage fd-tet
  MISC_FEATURE (1)..(1): X = H, R or K
XXXXH

(19) United States
(12) Patent Application Publication
(10) Pub. No.: US 2006/0039858 A1
(43) Pub. Date: Feb. 23, 2006

(54) Radiolabeled antibodies and peptides for treatment of tumors

(76) Inventors: Ekaterina Dadachova, Mahopac, NY (US); Joshua D. Nosanchuk, Upper Saddle River, NJ (US); Arturo Casadevall, Pelham, NY (US)

Correspondence Address: AMSTER, ROTHSTEIN & EBENSTEIN LLP, 90 PARK AVENUE, NEW YORK, NY, 10016, US

(21) Appl. No.: 11/201,394

(22) Filed: Aug. 10, 2005

Related U.S. Application Data
(63) Continuation-in-part of application No. 10/775,869, filed on Feb. 10, 2004.
(60) Provisional application No. 60/446,684, filed on Feb. 11, 2003.

(51) Int. Cl. A61K 51/00; C12Q 1/68

(52) U.S. Cl. 424/1.49; 435/6; 424/1.69

(57) ABSTRACT

This invention provides methods for imaging and/or treating a tumor in a subject which comprise administering to the subject an amount of a radiolabeled antibody and/or peptide effective to image and/or treat the tumor, where the radiolabeled antibody and/or peptide binds to a cellular component released by dying tumor cells. This invention also provides methods for imaging and/or treating melanin-containing melanomas or other melanin-containing tumors in a subject which comprise administering to the subject an amount of a radiolabeled anti-melanin antibody and/or peptide effective to image and/or treat the melanoma or tumor. The invention also provides compositions and methods of making compositions comprising radiolabeled antibodies and/or peptides for imaging and treating tumors, including melanin-containing melanomas.

### CROSS-REFERENCE TO RELATED APPLICATIONS

This application claims priority of and is a continuation-in-part of U.S. patent application Ser. No. 10/775,869, filed Feb. 10, 2004, which claims the benefit of U.S. Provisional Patent Application No. 60/446,684, filed Feb. 11, 2003, the contents of both of which are hereby incorporated by reference in their entirety into the subject application.

### STATEMENT OF GOVERNMENT SUPPORT

The invention disclosed herein was made with U.S. Government support under grant numbers A1001489 and A152733 from the National Institutes of Health, U.S. Department of Health and Human Services. Accordingly, the U.S. Government has certain rights in this invention.

### FIELD OF THE INVENTION

The present invention relates to the imaging and treatment of melanin-containing melanomas using radiolabeled anti-melanin antibodies and radiolabeled anti-melanin peptides, and to the imaging and treatment of tumors using radiolabeled antibodies and peptides that bind to a cellular component released by dying tumor cells.

### BACKGROUND OF THE INVENTION

Throughout this application various publications are referred to in parenthesis. Full citations for these references may be found at the end of the specification immediately preceding the claims. The disclosures of these publications are hereby incorporated by reference in their entireties into the subject application to more fully describe the art to which the subject application pertains.

There is a clinical need for new therapies for melanoma which is among the few cancers with a rising incidence (1). Malignant melanoma affects ˜40,000 new patients each year in the United States and an estimated 100,000 world-wide (2, 3). Melanoma is an important cause of cancer among young patients (30-50 years) which increases the economic importance of the disease. While primary tumors are successfully removed surgically, a satisfactory treatment for patients with metastatic melanoma has not been developed (4). The median survival time of patients with metastatic melanoma is 8.5 months, with an estimated 5-year survival of 6% (4). There has been little change in these results over the past 25 years.

Immune approaches to the therapy of metastatic melanoma have been evolving steadily and include treating patients with 1) non-specific immune stimulants with a focus on the use of tumor-associated antigens by passive immune therapy with antibodies targeted directly to tumor cells; and 2) active immune therapy via vaccination with tumor cells, tumor cell lysates, peptides, carbohydrates, gene constructs encoding proteins, or anti-idiotype antibodies that mimic tumor-associated antigens (5).

Monoclonal antibodies (mAbs) radiolabeled with diagnostic radioisotopes 99m-Technetium (^{99m}Tc) and 111-Indium (¹¹¹In) as well as with 131-Iodine (¹³¹I) have been used extensively for radioimmunoimaging (RII) of metastatic melanoma. A recent review by Kang and Yong (6) summarizes 58 patient trials (excluding case studies) involving a total of 3638 patients. The majority (>80%) of these studies used mAbs to high molecular weight melanoma associated antigen (HMW-MAA) proteoglycan. The sensitivity of RII using various anti-HMW-MAA mAbs or mAbs against other melanoma associated antigens such as P97 (7) is 65-88% (5, 6) which compares favorably with standard diagnostic methods (6). RI is also able to survey the entire body for metastases in a single study and can detect a substantial number of otherwise occult lesions.

Although RII has filled a niche in detection and disease assessment of metastatic melanoma, the ultimate goal is radioimmunotherapy (RIT). RIT takes advantage of the specificity of the antigen-antibody interaction to deliver lethal doses of radiation to target cells using radiolabeled antibodies (8). RIT is experiencing a renaissance, and so far has been most successful for the treatment of “liquid” and “semi-liquid” malignancies such as lymphoma and leukemia (9). The recent FDA approval of Zevalin® (IDEC Pharmaceuticals, San Diego, Calif.), which is 90-Yttrium (⁹⁰Y) labeled monoclonal anti-CD20 antibody for treatment of relapsed or refractory B-cell non-Hodgkin's lymphoma is proof of the enormous potential of RIT in cancer treatment.

There have been relatively few attempts to use RIT for treatment of melanoma in either the pre-clinical or clinical settings. One possible explanation for this might be the perception of melanoma as a relatively radioresistant cancer (10, 11) resulting from the outcomes of radiation therapy of melanoma with external beam radiation. Radioresistance in melanoma has been associated with melanin contents which presumably provide a non-specific shield that absorbs photons. The perception that melanoma is radioresistant is changing now (11) and, more importantly, it has been shown that radioresistance of certain tumors towards external radiation beam is higher compared to treatment of the same type of tumors with radioimmunotherapy. The difference in efficacy is due to different mechanisms of interaction between tumor cells and gamma rays of external beam compared to the particulate radiation delivered by radiolabeled antibodies (12, 13). Significant killing of melanoma cells in monolayers was observed as a result of treatment with antibodies radiolabeled with 125-Iodine (14), 211-Astatine (15), and 111-Indium (16). 131-I-labeled mAb caused shrinkage of human malignant melanoma multicellular spheroids (17). In an animal model of human melanoma, intratumoral injection of mAb radiolabeled with alpha-emitter 213-Bismuth caused complete disappearance of xenografted tumors while systemic RIT was less efficient with some delay in tumor progression followed by eventual re-growth (18). In a pilot study in patients with metastatic melanoma (19), a patient who received total dose of 374 mCi 131-I-labeled Fab′ fragments of anti-HMW-MAA mAb showed a greater than 50% reduction in the size of pelvic and pericaval nodes, with stabilization of disease at the smaller nodal size for a period of several months.

The majority of human melanomas are pigmented with melanin. Although several antibodies have been tried for the therapy of melanomas (notably monoclonal antibodies against high molecular weight melanoma-associated antigen, against chondroitin sulfate proteoglycan, and against transferrin receptor), the approach of targeting melanin with an anti-melanin antibody has not been utilized. One factor which teaches away from the use of anti-melanin antibodies is that melanin is an intracellular pigment that is normally found in the melanosome. Hence, one might dismiss this pigment as a target as being inaccessible to a serum antibody. Another factor is that the amount of intracellular melanin is inversely related to the radiosensitivity of human melanoma cells (20-22). Melanin is thought to absorb radiation and thereby protect the cells.

### SUMMARY OF THE INVENTION

The present invention is directed to the use of melanin as an antigen for radioimmunotherapy (RIT) of melanoma and other melanin-containing tumors with radiolabeled anti-melanin antibodies and/or radiolabeled anti-melanin peptides. Targeting melanin with radiolabeled antibodies or peptides allows the use of RIT against tumors such as melanomas that contain melanin, e.g. pigmented melanomas and hypomelanotic melanomas which are the most common types of melanoma. Accordingly, the invention provides a method for treating a melanin-containing melanoma and other melanin-containing tumors in a subject which comprises administering to the subject an amount of a radiolabeled anti-melanin antibody or peptide effective to treat the melanoma or melanin-containing tumor. The invention also provides a method for imaging a melanin-containing melanoma or other melanin-containing tumor in a subject which comprises administering to the subject an amount of a radiolabeled anti-melanin antibody or peptide effective to image the melanoma or tumor.

This invention further provides methods of treating and/or imaging a tumor in a subject which comprise administering to the subject an amount of a radiolabeled antibody or peptide effective to treat and/or image the tumor, where the radiolabeled antibody or peptide binds to a cellular component released by a dying tumor cell.

The invention also provides a method of making a composition effective to treat or image a melanin-containing melanoma or other melanin-containing tumor in a subject which comprises admixing a radiolabeled anti-melanin antibody or peptide and a carrier. The invention further provides a composition comprising an amount of a radiolabeled anti-melanin antibody or peptide effective to treat and/or image a melanin-containing melanoma or other melanin-containing tumor in a subject and a carrier.

### BRIEF DESCRIPTION OF THE FIGURES

FIG. 1A-1B. Binding of ²¹³Bi-CHXA″-6D2 to SK-MEL-28 cells (A) and to MNT1 cells (B).

FIG. 2A-2C. Scintigraphic images of nude mice at 3 hours post-injection with: A) ¹¹¹In-6D2, administered IP to mice following IP injection of 2.8×10⁶ SK-28-MEL cells 24 hours earlier; B) ¹¹¹In-IgM, administered IP to mice following IP injection of 2.8×10⁶ SK-28-MEL cells 24 hours earlier; C) ¹¹¹In-6D2, administered IP to non-tumor-bearing mice.

FIG. 3A-3B. Scintigraphic images of MNT1 tumor-bearing nude mouse at 3 hours (A) and 24 hours (B) post-injection with ^{99m}Tc-6D2 antibody.

FIG. 4. Immunogold TEM of MNT1 melanoma cell stained with mAb 6D2 [original magnification ×20,000]. Left upper corner inset is the magnification of the central area of the melanoma cell indicated by the white box near the center of the image; right lower corner inset is the magnification of the extracellular area indicated by the black box in the upper right corner of the image. Arrows indicate gold balls labeling melanin.

FIG. 5. MNT1 human melanoma tumor growth in nude mice treated with: 1.5 mCi ¹⁸⁸Re-6D2; 1.5 mCi ¹⁸⁸Re-IgM; and 100 μg unlabeled 6D2.

FIG. 6A-6B. Scintigraphic images of radiolabeled 6D2 mAb in black and white mice: A) 3 h image of ¹⁸⁸Re-6D2 given IV to a black C57BL/6 mouse; B) 3 h image of ¹⁸⁸Re-6D2 given IV to a white BALB/c mouse. The positions of the tails are marked with arrows.

FIG. 7A-7B. Histological analysis of melanin-containing tissues of black C57BL/6 mice treated with ¹⁸⁸Re-6D2 antibody: A—eye of a mouse treated with 1.5 mCi ¹⁸⁸Re-6D2; B—eye of a control mouse.

FIG. 8A-8C Binding of 4B4 peptide to melanoma cells. A) Immunofluorescence of viable (arrow) and non-viable (arrowhead) MNT1 melanoma cells in vitro [original magnification ×250]. Left and right panels show light microscopy and immunofluorescence of MNT1 melanoma cells stained with 4B4 peptide, respectively. Only non-viable cells are stained consistent with the fact that cellular disruption is needed for peptide access to cellular melanin. B) Binding of ¹⁸⁸Re-HYNIC-4B4 to SK-MEL-28 whole and lysed cells. C) Binding of ¹⁸⁸Re-HYNIC-4B4 to MNT1 whole and lysed cells. For control the cells were pre-incubated with excess (2 μg/mL) of HYNIC-4B4. The cells were lysed before addition of ¹⁸⁸Re-HYNIC-4B4.

FIG. 9A-9C. HPLC analysis of oxidation products of melanin from different melanoma cell lines: A) background solution; B) SK-MEL-28 cells; C) MNT1 cells.

FIG. 10A-10B. Tissue distribution of ¹⁸⁸Re-HYNIC-4B4. A) MNT1 tumors-bearing nude mice. Four mice per group were used. B) White BALB/c and black C57BL6 mice. Five mice per group were used. In both experiments mice were injected IV with 2 FIG. ¹⁸⁸Re-HYNIC-4B4 (50 μCi).

FIG. 11A-11B. Therapy of MNT1 pigmented melanoma tumors in nude mice with ¹⁸⁸Re-HYNIC-4B4 peptide. Points represent averages of tumor size from 10 mice. The bars represent standard deviation: A) 1st study in mice with 0.5-0.7 cm tumors; B) 2nd study in mice with 0.3-0.4 cm tumors. “¹⁸⁸Re-decapeptide” is ¹⁸⁸Re-labeled irrelevant decapeptide HYNIC-PA1.

FIG. 12A-12G. Histological evaluation of ¹⁸⁸Re-HYNIC-4B4 effect on MNT1 tumor and normal tissues: A) large pleomorphic pigmented melanoma cells in control tumor (H&E); B) fibrosis, phagocytic histiocytes with intra- and extracellular melanin pigment are present but no residual neoplasm in treated tumor (H&E); C) fibrosis contains melanin pigment and single granule of iron (arrow), but no malignant cells in treated tumor (Prussian Blue); D) same area of tissue as B and C, but melanin pigment removed by bleaching to reveal bland fibrosis and histiocytes without evidence of malignant cells (melanin bleach); E) kidney glomerulus from treated tumor-bearing mice 3 months post-treatment (H&E); F) eye from C57BL6 mouse 2 months post-treatment (H&E); G) substantia nigra from C57BL6 mouse 2 months post-treatment (toluidene blue). All images have ×400 original magnification. All mice except for control were treated with 2×1 mCi ¹⁸⁸Re-HYNIC-4B4. The tumor tissue is from the mice used in 2nd therapy study.

FIG. 13. Schematic of the conceptual approach to radiotherapy by anti-melanin antibody or peptide. Melanin is released from melanoma cells as a consequence of cell turnover. Anti-melanin antibody or anti-melanin peptide binds to free melanin and delivers cytotoxic radiation to the area. Melanized, weakly melanized and amelanotic cells are killed by radiation through “cross-fire” effect.

### DETAILED DESCRIPTION OF THE INVENTION

This invention provides a method of treating a tumor in a subject which comprises administering to the subject an amount of a radiolabeled antibody and/or radiolabeled peptide effective to treat the tumor, where the radiolabeled antibody or peptide binds to a cellular component released by a dying tumor cell. The invention also provides a method of imaging a tumor in a subject which comprises administering to the subject an amount of a radiolabeled antibody and/or peptide effective to image the tumor, where the radiolabeled antibody or peptide binds to a cellular component released by a dying tumor cell. The cellular component can be a histone, a mitochondrial protein, a cytoplasmic protein, or a pigment, e.g. melanin. The histone can be one of the major subtypes of histones, i.e. H1, H2A, H2B, H3 and H4. In one embodiment, the tumor is a melanoma and the cellular component is melanin, in which case a melanin-binding antibody can be referred to as an anti-melanin antibody and a melanin-binding peptide can be referred to as an anti-melanin peptide.

The subject invention is also directed to a method for treating a melanin-containing melanoma or other melanin-containing tumor in a subject which comprises administering to the subject an amount of a radiolabeled anti-melanin antibody and/or a radiolabeled anti-melanin peptide effective to treat the melanoma or melanin-containing tumor. The invention further provides a method for imaging a melanin-containing melanoma or other melanin-containing tumor in a subject which comprises administering to the subject an amount of a radiolabeled anti-melanin antibody and/or peptide effective to image the melanoma or tumor. In addition to melanomas, examples of melanin-containing tumors include pigmented schwannomas and pigmented neurofibromas.

As used in the subject application, the term “antibody” encompasses whole antibodies and fragments of whole antibodies. Antibody fragments include, but are not limited to, F(ab′)₂ and Fab′ fragments. F(ab′)₂ is an antigen binding fragment of an antibody molecule with deleted crystallizable fragment (Fc) region and preserved binding region. Fab′ is ½ of the F(ab′)₂ molecule possessing only ½ of the binding region. The term antibody is further meant to encompass polyclonal antibodies and monoclonal antibodies. In one embodiment, the antibody fragment or peptide specifically binds to a cellular component released by a dying tumor cell. In one embodiment, the antibody fragment or peptide specifically binds to melanin.

The antibody can be any of an IgA, IgD, IgE, IgG, or IgM antibody. The IgA antibody can be an IgA1 or an IgA2 antibody. The IgG antibody can be an IgG1, IgG2, IgG2a, IgG2b, IgG3 or IgG4 antibody. A combination of any of these antibodies can also be used. One consideration in selecting the type of antibody to be used is the desired serum half-life of the antibody. IgG has a serum half-life of 23 days, IgA 6 days, IgM 5 days, IgD 3 days, and IgE 2 days (60). Another consideration is the size of the antibody. For example, the size of IgG is smaller than that of IgM allowing for greater penetration of IgG into tumors. IgA, IgG, and IgM are preferred antibodies.

In one embodiment, the antibody is 6D2. In one embodiment, the antibody is an antibody generated against human melanin.

Melanin-binding peptides have been described, where the melanin-binding peptide is a decapeptide (24). In one embodiment, the melanin-binding decapeptide is 4B4 (YERKFWHGRH) (SEQ ID NO:1). Melanin-binding peptides longer or shorter than 10 amino acids can also be used. Important structural characteristic of melanin-binding peptides are the presence of aromatic amino acids and overall positive charge, e.g. in water at a pH value of 7.2-7.4. Additional melanin-binding peptides include peptides that contain the amino acid sequence (YERKFWHGRH) (SEQ ID NO:1), LHKLVRHGRW (SEQ ID NO:2), YLRRHTHVFW (SEQ ID NO:3), KKHSHYWVRY (SEQ ID NO:4), EFGTRHMRHR (SEQ ID NO:5), YRHHAHGGRG (SEQ ID NO:6), RKKWHGWTRW (SEQ ID NO:7), PKWRHGYTRF (SEQ ID NO:8), RHGTVKHARH (SEQ ID NO:9), RRHWHPPVQI (SEQ ID NO:10), EAYKRRWHWP (SEQ ID NO:11), RWPKRHLSGH (SEQ ID NO:12), SRVPFRHYHH (SEQ ID NO:13), RRPEHTKARW (SEQ ID NO:14), WRAFLPRWHA (SEQ ID NO:15), WNRGWRWWMG (SEQ ID NO:16), GFFWKWRIGR (SEQ ID NO:17), and HIRWKGHISW (SEQ ID NO:18). Preferred melanin-binding peptides comprise the amino acid sequence motif X₁-X₂-X₃-X₄-H (SEQ ID NO:19), where X₁ and X₂ are positively charged amino acids, and X₃ and X₄ are positively charged amino acids and/or aromatic amino acids. Lysine (K or Lys), arginine (R or Arg), and histidine (H or His) are positively charged amino acids. Aromatic amino acids include histidine, phenylalanine (F or Phe), tyrosine (Y or Tyr), and tryptophan (W or Try). A preferred melanin-binding peptide contains one, two or more (one or more) of the above amino acid motifs that are preferably spaced apart by one to three residues, although the motifs can also overlap when X₁ is H so that the last residue of a first motif is the first residue of a second motif. It is also preferred that a contemplated melanin-binding peptide be comprised of at least one and preferably all D, rather than the usual L, amino acid residues. It is unexpectedly found that contrary to usual binding phenomena involving proteinaceous materials, such as these peptides, in which L-amino acid residues must be used, D-amino acid residues can be used here, and peptides prepared from those D-amino acid residues can survive in vivo for a greater time than corresponding peptides made from L-amino acid residues.

As used herein, the term “tumor” includes melanomas. The term “treat” a tumor means to eradicate the tumor, to reduce the size of the tumor, to stabilize the tumor so that it does not increase in size, or to reduce the further growth of the tumor.

The subject can be a mammal. In different embodiments, the mammal is a mouse, a rat, a cat, a dog, a horse, a sheep, a cow, a steer, a bull, livestock, a primate, a monkey, or preferably a human.

The choice of the particular radioisotope with which the antibody or peptide is labeled may be determined by the size of the tumor to be treated and its localization in the body. Two characteristics are important in the choice of a radioisotope—emission range in the tissue and half-life. Alpha emitters, which have a short emission range in comparison to beta emitters, may be preferable for treatment of small tumors or melanomas that are disseminated in the body. Examples of alpha emitters include 213-Bismuth (half-life 46 minutes), 223-Radium (half-life 11.3 days), 224-Radium (half-life 3.7 days), 225-Radium (half-life 14.8 days), 225-Actinium (half-life 10 days), 212-Lead (half-life 10.6 hours), 212-Bismuth (half-life 60 minutes), 211-Astatin (half-life 7.2 hours), and 255-Fermium (half-life 20 hours). In a preferred embodiment, the alpha-emitting radioisotope is 213-Bismuth). Bi emits a high LET α-particle with E=5.9 MeV with a path length in tissue of 50-80 μm. Theoretically a cell can be killed with one or two α-particle hits. ²¹³Bi has been proposed for use in single-cell disorders and some solid cancers (34, 35-37) and has been used to treat patients with leukemia in Phase I clinical trials (38, 39). ²¹³Bi is the only α-emitter that is currently available in generator form, which allows transportation of this isotope from the source to clinical centers within the United States and abroad.

Beta emitters, with their longer emission range, may be preferable for the treatment of large tumors or melanomas. Examples of beta emitters include 188-Rhenium (half-life 16.7 hours), 90-Yttrium (half-life 2.7 days), 32-Phosphorous (half-life 14.3 days), 47-Scandium (half-life 3.4 days), 67-Copper (half-life 62 hours), 64-Copper (half-life 13 hours), 77-Arsenic (half-life 38.8 hours), 89-Strontium (half-life 51 days), 105-Rhodium (half-life 35 hours), 109-Palladium (half-life 13 hours), 111-Silver (half-life 7.5 days), 131-Iodine (half-life 8 days), 177-Lutetium (half-life 6.7 days), 153-Samarium (half-life 46.7 hours), 159-Gadolinium (half-life 18.6 hours), 186-Rhenium (half-life 3.7 days), 166-Holmium (half-life 26.8 hours), 166-Dysprosium (half-life 81.6 hours), 140-Lantanum (half-life 40.3 hours), 194-Irridium (half-life 19 hours), 198-Gold (half-life 2.7 days), and 199-Gold (half-life 3.1 days). In a preferred embodiment, the beta-emitting radioisotope is 188-Rhenium. ¹⁸⁸Re is a high-energy β-emitter (Emax=2.12 MeV) that has recently emerged as an attractive therapeutic radionuclide in diverse therapeutic trials including cancer radioimmunotherapy, palliation of skeletal bone pain, and endovascular brachytherapy to prevent restenosis after angioplasty (31-33). ¹⁸⁸Re has the additional advantage that it emits γ-rays which can be used for imaging studies. For the treatment of large tumors or melanomas or those in difficult to access sites deep in the body, longer-lived isotopes such as 90-Yttrium (half-life 2.7 days), 177-Lutetium (half-life 6.7 days) or 131-Iodine (half-life 8 days) may be preferred.

Positron emitters could also be used, such as (half-life in parenthesis): ^{52m}Mn (21.1 min); ⁶²Cu (9.74 min); ⁶⁸Ga (68.1 min); ° C. (20 min); ⁸²Rb (1.27 min); ¹¹⁰In (1.15 h); ¹¹⁸Sb (3.5 min); ¹²²I (3.63 min); ¹⁸F (1.83 h); ^{34m}Cl (32.2 min); ³⁸K (7.64 min); ⁵¹Mn (46.2 min); ⁵²Mn (5.59 days); ⁵²Fe (8.28 h); ⁵⁵Co (17.5 h); ⁶¹Cu (3.41 h); ⁶⁴Cu (12.7 h); ⁷²As (1.08 days); ⁷⁵Br (1.62 h); ⁷⁶Br (16.2 h); ^{82m}Rb (6.47 h); ⁸³Sr (1.35 days); ⁸⁶Y (14.7 h); ⁸⁹Zr (3.27 days); ^{94m}Tc (52.0 min); ¹²⁰I (1.35 h); ¹²⁴I (4.18 days). 64-Copper is a mixed positron, electron and Auger electron emitter.

Any of the radioisotopes, except alpha emitters, that are used for radioimmunotherapy can also be used at lower doses for radioimmunoimaging, for example a beta emitter, a positron emitter or an admixture of a beta emitter and a positron emitter. Preferred radioisotopes for use in radioimmunoimaging include 99m-Technetium, 111-Indium, 67-Gallium, 123-Iodine, 124-Iodine, 131-Iodine and 18-Fluorine. For imaging one can use a dose range of 1-30 mCi for diagnostic isotopes (e.g., 99m-Tc) and 1-5 mCi for therapeutic isotopes to avoid unnecessary dose to a patient.

The invention further provides methods for treating tumors or melanin-containing melanoma in a subject which comprise administering to the subject an amount of antibodies and/or peptides radiolabeled with a plurality of different radioisotopes effective to treat the tumor. Preferably, the radioisotopes are isotopes of a plurality of different elements. In a preferred embodiment, at least one radioisotope in the plurality of different radioisotopes is a long range emitter and at least one radioisotope is a short range emitter. Examples of long range emitters include beta emitters and positron emitters. Examples of short range emitters include alpha emitters. Positron emitters can also be intermediate range emitters depending on the energy of the positrons. In a preferred embodiment, the long-range emitter is a beta emitter and the short range emitter is an alpha emitter. Preferably, the beta emitter is 188-Rhenium. Preferably, the alpha emitter is 213-Bismuth. Combinations of different radioisotopes can be used, which include an admixture of any of an alpha emitter, a beta emitter, and a positron emitter, with physical half-lives from 30 minutes to 100 days. Preferably, the plurality of different radioisotopes is more effective in treating the tumor than a single radioisotope within the plurality of different radioisotopes, where the radiation dose of the single radioisotope is the same as the combined radiation dose of the plurality of different radioisotopes.

It is known from radioimmunotherapy studies of tumors that whole antibodies usually require from 1 to 3 days time in circulation to achieve maximum targeting. While slow targeting may not impose a problem for radioisotopes with relatively long half-lives such as ¹⁸⁸Re (t_{1/2}=16.7 hours), faster delivery vehicles are needed for short-lived radioisotopes such as ²¹³Bi (t_{1/2}=46 min). The smaller melanin-binding peptides and F(ab′)₂ and Fab′ fragments provide much faster targeting which matches the half-lives of short-lived radionuclides (55, 56).

The dose of the radioisotope can vary depending on the localization and size of the tumor, the method of administration of radiolabeled antibody (local or systemic) and the decay scheme of the radioisotope. In order to calculate the doses which can treat the tumor without radiotoxicity to vital organs, a diagnostic scan of the patient with the antibody radiolabeled with a diagnostic radioisotope or with a low activity therapeutic radioisotope can be performed prior to therapy, as is customary in nuclear medicine. The dosimetry calculations can be performed using the data from the diagnostic scan (59). In different embodiments, the dose of the radioisotope for RIT is between 1-1000 mCi.

Clinical data (39, 58) indicate that fractionated doses of radiolabeled antibodies and peptides are more effective than single doses against tumors and are less radiotoxic to normal organs. Depending on the status of a patient and the effectiveness of the first treatment with RIT, the treatment may consist of one dose or several subsequent fractionated doses.

The uptake of radiolabeled antibody or peptide by the kidney can be reduced or inhibited by administering a positively charged amino acid to the subject (29, 30), such as lysine, arginine or histidine. A preferred amino acid is D-lysine.

Preferably, the uptake of radiolabeled anti-melanin antibody or peptide in the melanoma or radiolabeled antibody or peptide in the tumor is at least 10 times greater than in surrounding muscle. Preferably, the radiolabeled anti-melanin antibody or peptide is not taken up by non-cancerous (i.e., normal or healthy) melanin-containing tissue, including, but not limited to, hair, eyes, skin, brain, spinal cord, and/or peripheral neurons.

The invention provides a method of using a radiolabeled anti-melanin antibody or peptide to image and/or treat a melanin-containing melanoma or other melanin-containing tumor in a subject which comprises:
(a) generating a peptide or a monoclonal antibody against melanin;
(b) attaching a radiolabel to the peptide or monoclonal antibody; and
(c) administering to the subject an amount of the radiolabeled antibody or peptide effective to image and/or treat the melanoma.

The invention further provides a method of using a radiolabeled antibody or peptide to image and/or treat a tumor in a subject which comprises:
(a) generating a peptide or a monoclonal antibody against a cellular component released by a dying tumor cell;
(b) attaching a radiolabel to the peptide or monoclonal antibody; and
(c) administering to the subject an amount of the radiolabeled antibody or peptide effective to image and/or treat the tumor.

Antibodies can be readily generated without undue experimentation using the protocol given below in Experimental Details.

The invention provides a method of making a composition effective to treat a melanin-containing melanoma or other melanin-containing tumor in a subject which comprises admixing a radiolabeled anti-melanin antibody and/or a radiolabeled anti-melanin peptide and a carrier. The invention also provides a method of making a composition effective to image a melanin-containing melanoma or other melanin-containing tumor in a subject which comprises admixing a radiolabeled anti-melanin antibody and/or peptide and a carrier. The invention further provides a method of making a composition effective to image and/or treat a tumor in a subject which comprises admixing a radiolabeled antibody and/or peptide and a carrier, where the antibody or peptide binds to a cellular component released by a dying tumor cell. The invention provides a composition made by any of these methods, i.e. a composition comprising an anti-melanin antibody or peptide effective to treat and/or image a melanin-containing melanoma in a subject and a carrier. As used herein, the term “carrier” encompasses any of the standard pharmaceutical carriers, such as a sterile isotonic saline, phosphate buffered saline solution, water, and emulsions, such as an oil/water emulsion.

The melanin-containing melanoma can be, for example, a pigmented melanoma, a hypomelanotic melanoma, or an amelanotic melanoma. So-called “amelanotic melanomas” are generally hypomelanotic and contain small amounts of melanin (61, 62). Other melanin-containing tumors include pigmented schwannomas and pigmented neurofibromas (76).

This invention will be better understood from the Experimental Details which follow. However, one skilled in the art will readily appreciate that the specific methods and results discussed are merely illustrative of the invention as described more fully in the claims which follow thereafter.

### Experimental Details

1. Introduction

Melanin is an intracellular pigment that is normally found in the melanosome. Hence, one might dismiss this pigment as a target for a serum antibody because melanin could be expected to be inaccessible. However, melanomas, like many rapidly growing tumors, can be assumed to have a large cell turnover resulting in cell lysis and release of pigment. Hence, an antibody or peptide to melanin can bind to melanin by virtue of the presence of extracellular pigment originating from dead cells. Furthermore, since most of the melanin in the body in healthy tissue is intracellular, it is believed that antibody- or peptide-therapy targeting melanin would not harm pigmented cells such as normal melanocytes or melanin-containing neurons.

Monoclonal antibodies have been developed against fungal melanin produced by C. neoformans. These mAbs bind to melanin produced by other microorganisms such as Sepia officinalis as well as to synthetic melanin (23-25). Since both fungal melanin and melanin in tumors are negatively charged and reagents generated to fungal melanin recognize melanin from diverse sources (25, 26), it was hypothesized that fungal melanin-binding antibody would bind to melanin in melanoma cells and would be able to deliver radioisotopes to the tumors in vivo.

2. Materials and Methods

Radiolabeled anti-melanin antibodies. An anti-melanin antibody (mAb 6D2) that was originally developed against fungal melanin (23) was used as a carrier delivery vehicle to deliver therapeutic radioactivity (e.g. radioimmunotherapy (RIT)) to a pigmented melanoma. MAb 6D2 (IgM type) was generated from hybridomas obtained from mice immunized with melanin isolated from Cryptococcus neoformans. Na^{99m}TcO₄ was purchased from Syncor (Bronx, N.Y.), and ¹¹¹InCl₃ from Iso-Tex Diagnostics, Friendswood, Tex. ¹⁸⁸Re in the form of Na perrhenate (Na¹⁸⁸ReO₄) was eluted from a ¹⁸⁸W/¹⁸⁸Re generator (Oak Ridge National Laboratory (ORNL), Oak Ridge, Tenn.). 225-Actiunium (²²⁵Ac) for construction of a ²²⁵AC/²¹³Bi generator was acquired from ORNL. The ²²⁵AC/²¹³Bi generator was constructed using a MP-50 cation exchange resin, and ²¹³Bi was eluted with 0.15 M HI (hydroiodic acid) in the form of ²¹³BiI₃ as described in (52). The mAb was radiolabeled with ²¹³Bi (213-Bismuth, short-range alpha-emitting isotope for therapy) or ¹¹¹In (111-Indium, photon emitter for imaging use, chemical analogue of ²¹³Bi) via the bifunctional chelator CHXA″ (N-[2-amino-3-(p-isothiocyanatophenyl)propyl]-trans-cyclobexane-1,2-diamine-N,N′,N″,N′″,N″″-pentaacetic acid) (57); and with ¹⁸⁸Re (188-Rhenium, long-range beta-emitting isotope for therapy) and ^{99m}Tc (99m-Technetium, photon emitter for imaging, chemical analogue of ¹⁸⁸Re) via “direct labeling” through reduction of antibody disulfide bonds with dithiothreitol (54). The immunoreactivity of radiolabeled 6D2 mAb towards fungal melanin was tested by immunofluorescence.

Melanin-binding peptide synthesis and radiolabeling. Fungal melanin-binding peptides have been previously identified and sequenced from a phage display library (24). The melanin-binding decapeptide 4B4 (YERKFWHGRH) (SEQ ID NO:1) was synthesized from D-amino acids with N terminal biotin labeling in the Laboratory for Macromolecular Analysis and Proteomics (Albert Einstein College of Medicine, Bronx, N.Y.). For labeling with ¹⁸⁸Re, the 4B4 peptide and irrelevant control decapeptide PA1 (24) were synthesized from D-amino acids with HYNIC (hydrazinonicotinamide) ligand at the N terminus using Fmoc reagent 6-Fmoc-hydrazino-nicotinic acid (Trilink Biotechnology, Inc.). The molecular mass of ¹⁸⁸Re-HYNIC-4B4 determined by mass spectrometry was 1550.

¹⁸⁸Re in the form of sodium perrhenate Na¹⁸⁸ReO₄ was eluted from a ¹⁸⁸W/¹⁸⁸Re generator (Oak Ridge National Laboratory, Oak Ridge, Tenn.) and HYNIC-4B4 and HYNIC-PA1 peptides were radiolabeled with ¹⁸⁸Re-gluconate by incubation for 1 h at room temperature while protected from light according to (53). Incorporation of radioactivity into the peptides was determined by instant thin layer chromatography with silica gel-impregnated glass fibers (ITLC-SG) developed with saline. In this system, ¹⁸⁸Re-labeled peptides had an R_{f}=0 while ¹⁸⁸Re-gluconate and ¹⁸⁸Re-perrhenate moved with the solvent front. If needed, the radiolabeled peptides were purified on SEP-PAK18 chromatographic column as described in (64).

Serum stability of ¹⁸⁸Re-HYNIC-4B4 and ¹⁸⁸Re-HYNIC-PA1. ¹⁸⁸Re-HYNIC-4B4 was incubated in mouse serum at 37° C., and aliquots were withdrawn at 0, 0.5, 1, 2, 3, 4 and 5 hours and analyzed on size exclusion HPLC column eluted with PBS, pH 7.2 at 1 mL/min. Peptide and proteins were monitored by UV detector at 280 nm; 1 mL fractions were collected and counted in a dose calibrator.

Melanoma cells. Human lightly pigmented melanoma cells SK-MEL-28 (ATCC) were grown in complete growth medium (ATCC) supplemented with 10% FBS and 110 μM L-tyrosine to promote melanin formation. Highly pigmented human melanoma cells MNT1 (27) were grown in MEM/20% FBS medium. The percentage of viable cells in the samples was determined to be 96±1% by Trypan blue exclusion assay.

Binding of ¹⁸⁸Re-HYNIC-4B4 to SK-MEL-28 and MNT1 cells. The binding of ¹⁸⁸Re-HYNIC-4B4 to SK-MEL-28 and MNT1 cells was evaluated by incubating labeled peptide (20 ng/mL) with 0.2-2.0×10⁶ cells. Peptide binding to both whole and osmotically lysed cells was evaluated. After incubation for 1 h at 37° C. the cells were collected by centrifugation, the supernatant was removed, the cell pellet washed with PBS, and the pellet and the supernatant were counted in a gamma counter to calculate the percentage of peptide binding to the cells. To prove that the binding of peptide was specific, cells were also pre-incubated with an excess (2 μg/mL) of unlabeled HYNIC-4B4.

Immunofluorescence of MNT1 cells. The binding of the 4B4 peptide to melanoma cells in vitro was analyzed by immunofluorescence as in (24). Approximately 106 melanoma cells were blocked for non-specific binding by incubation in SuperBlock (Pierce, Rockford, Ill.) for 1 h at 37° C. Biotinylated 4B4 was then incubated with the cells for 1 h followed by addition of streptavidin conjugated with fluorescein isothiocyanate (FITC). The slides were viewed with an Olympus AX70 microscope (Melville, N.Y.) equipped with a FITC filter. Irrelevant biotinylated decapeptide PA1 (24) was used as a negative control.

HPLC analysis of melanin from SK-28-MEL and MNT1 melanoma cells. Melanin from MNT1 and SK-MEL-28 melanoma cells was purified using a modified methodology for isolating melanin from fungal cells (40). Briefly, the cells were subjected to the sequence of enzymatic digestion, boiling in 6 M HCl, extensive dialysis against deionized water and drying at 50° C. Purified melanin was subjected to acidic permanganate oxidation as described in (65, 66) and the oxidation products were analysed by HPLC using a Shimadzu LC-600 chromatography system, Hamilton PRP-1 C₁₈ column (250×4.1 mm dimensions, 7 μm particle size), and Shimadzu SPD-6AV UV detector. The mobile phase was 0.1% trifluoroacetic acid in water (solvent A) and 0.1% trifluoroacetic acid in acetonitrile (solvent B). At 1.0 mL/min, the elution gradient was (min, % B): 0, 0; 1, 0; 12, 25; 14, 25; 16, 0. The UV detector was set at a 255 nm absorbance. Pyrrole-2,3,5-tricarboxylic acid (PTCA),1,3-thiazole-2,4,5-tricarboxylic acid (TTCA) and 1,3-thiazole-4,5-dicarboxylic acid (TDCA) were used as standard compounds. Chromatograms of TDCA, TTCA and PTCA standards yielded peaks at 6.1, 7.1 and 11.0 min, respectively.

Animal models. All animal studies were carried out in accordance with the guidelines of the Institute for Animals Studies at the Albert Einstein College of Medicine. Biodistribution and therapy studies were carried out by injecting human melanoma cells into nude mice. The use of nude mice is essential to prevent the mouse immune system from clearing the human cells. In one set of experiments, mice were injected IP with 2.8×10⁶ human lightly pigmented melanoma line SK-28-MEL cells (ATCC) 24 hours before injections of radiolabeled antibody. In other experiments with radiolabeled antibody and in experiments with radiolabeled peptide, melanoma-like lesions were created in nude mice using highly pigmented human melanoma cells MNT1 (27). Tumors were induced by injecting approximately 5.5×10⁶ MNT1 cells into the right flank of female nude mice. The tumors reached 0.3-1 cm in diameter 4 weeks after implantation.

Comparative biodistribution of ¹⁸⁸Re-HYNIC-4B4 in the eyes and skin on the tails was performed in white BALB/c and black C57BL6 female mice, that have black eyes and melanized skin on their tails. Toxicity of therapeutic doses of ¹⁸⁸Re-HYNIC-4B4 to melanized normal tissues (eyes, skin and melanized neurons in substantia nigra) as well as to the brain was evaluated in C57BL6 female mice.

Biodistribution of ¹⁸⁸Re-HYNIC-4B4 in MNT1 tumor-bearing nude mice and in C57BL6 mice. To assess the uptake of ¹⁸⁸Re-HYNIC-4B4 peptide in the tumor and normal organs, MNT1 tumor-bearing nude mice were injected IV with 2 μg (50 μCi) ¹⁸⁸Re-HYNIC-4B4. Animals (4 mice per time interval) were sacrificed at 30 min, 1, 2, 3 and 24 h post-injection, their major organs removed, blotted to remove blood, weighted and counted in a gamma counter. Comparative biodistribution in the eyes and tail skin was similarly done in normal white BALB/c and black C57BL6 mice sacrificed 1 and 24 h after IV administration of 2 μg (50 μCi) ¹⁸⁸Re-HYNIC-4B4.

Therapy of MNT1 tumor-bearing mice with ¹⁸⁸Re-HYNIC-4B4. Two therapy experiments were conducted in MNT1 tumor-bearing mice. Ten animals per group were used in both studies. During the initial study, animals with tumors of 0.5-0.7 cm in diameter were used. Mice in the 1st group were treated IP with 1 mCi ¹⁸⁸Re-HYNIC-4B4 (2 μg), the 2nd group received 2×1 mCi ¹⁸⁸Re-HYNIC-4B4 20 days apart to investigate the effect of multiple treatments on tumor progression, and the 3rd group was left untreated. The follow-up study investigated the influence of tumor size on the therapy results as well as introduced another control in the form of ¹⁸⁸Re-labeled irrelevant decapeptide HYNIC-PA1. Mice with tumors 0.3-0.4 cm in diameter were used. Mice in the 1st group were treated IP with 2×1 mCi ¹⁸⁸Re-HYNIC-4B4 10 days apart, the 2nd group received 2×1 mCi ¹⁸⁸Re-HYNIC-PA1 10 days apart, and the 3rd group was left untreated. In both studies the size of the tumor was measured with calipers in 3 dimensions every 4 days and the tumor volume was calculated as the product of these measurements multiplied by 0.5. The animals were observed for 3 and 2 months post-treatment for tumor re-growth in the 1st and 2nd studies, respectively. To assess the effects of radiolabeled peptide on the tumor cells MNT1 tumors from ¹⁸⁸Re-HYNIC-4B4 treated and control mice were removed at the end of the 2nd study. Tumor tissues were fixed using 10% neutral buffered formalin, embedded in paraffin, cut and stained with hematoxylin and eosin (H&E). Prussian Blue stain was used to identify iron pigment. Masson-Fontana stain and melanin bleaching corroborated the H&E identification of melanin pigment.

Tumor and kidney dosimetry. For dosimetry calculations, the dosimetric model for a laboratory mouse was used, which takes into consideration self-doses for the organs and the cross-organ doses resulting from beta-radiation “cross-fire” (67). The biodistribution data was used to obtain cumulative activities by generating time-activity curves followed by integration of the area under the curve (Prism software, GraphPad, San Diego, Calif.).

Toxicity to the kidneys and normal melanized tissues post-treatment with ¹⁸⁸Re-labeled melanin-binding peptide. As nephrotoxicity after radiolabeled peptide therapy remains of concern (68), an assessment was made of the toxicity to the kidneys of the mice treated with 2×1 mCi ¹⁸⁸Re-HYNIC-4B4 and sacrificed at the conclusion of the 3 month study. To investigate whether radiation damage was induced by ¹⁸⁸Re-HYNIC-4B4 in melanin-containing normal tissues, five C57BL/6 received 2×1 mCi ¹⁸⁸Re-HYNIC-4B4 10 days apart. At 2 months after administration of ¹⁸⁸Re-HYNIC-4B4, mice were sacrificed and their eyes and melanized skin from the tails were removed. Tissues were prepared for histology as described above. Healthy nude and C57BL/6 mice were similarly studied for comparison.

Behavioral and histological assessment of toxicity to the brain. As peptides are small molecules that can potentially penetrate the blood brain barrier, and since the brain contains melanized tissues, the possibility of subtle damage that would manifest itself in behavioural changes was also considered. Consequently, behavioral assessments were performed of five C57BL/6 mice that received 2×1 mCi ¹⁸⁸Re-HYNIC-4B4 10 days apart and compared with five untreated controls. Afterwards histological evaluation of the subjects' substantia nigra was carried out to ascertain for the possibility of tissue damage. The behavioral assessment was done using the Primary screen SHIRPA Protocol (69), which is widely used for screening drug candidates by pharmaceutical laboratories. This method provides a behavioral and functional profile by observational assessment of mice and includes evaluation of gait, posture, motor control and coordination, changes in excitability and aggression, salivation, lacrimation, piloerection, muscle tone and temperature. It also tests for a gross measure of analgesia. All parameters are scored to provide a quantitative assessment that enables comparison of results over time. The behavior of treated and control animals was assessed 1 week and 2 month post-treatment with ¹⁸⁸Re-HYNIC-4B4. After the 2nd assessment with SHIRPA protocol, the mice were sacrificed by CO₂ asphyxiation and perfused through the heart with PBS followed by 4% paraformaldehyde (PFA). The brain was then extracted, fixed with 4% PFA overnight, paraffin embedded, sectioned, and the slides were stained with toluidine blue.

Statistics. The Wilcoxon Rank Sum test was used to compare organ uptake in biodistribution studies. Two-tailed Student's t test for unpaired data was employed to analyze differences in cell binding during in vitro studies and in tumor sizes during therapy studies. Differences were considered statistically significant when P values were <0.05.

3. Results

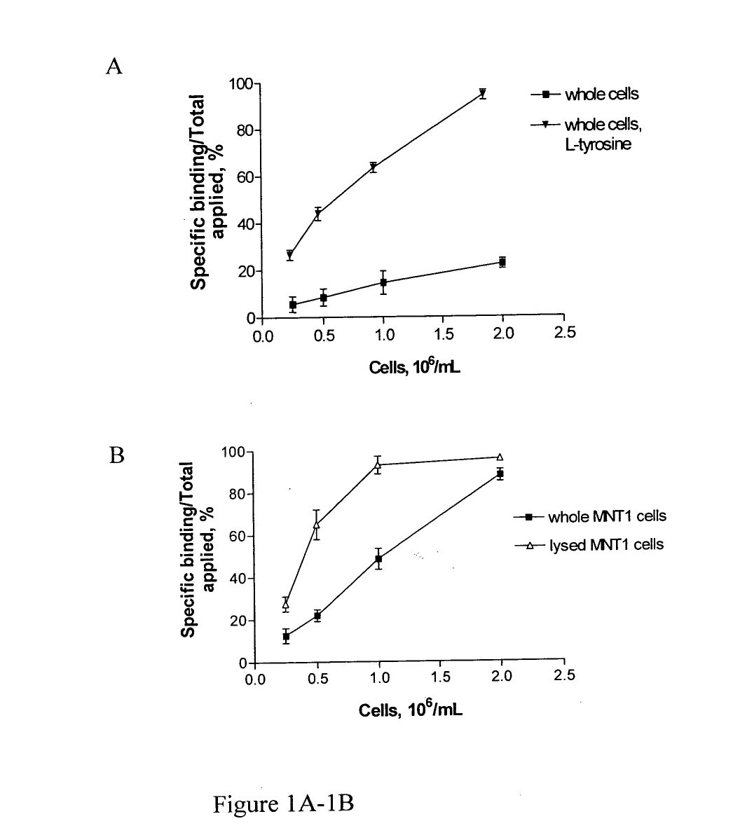

Binding of radiolabeled antibody to human pigmented melanoma cell line. Cell binding of ¹¹¹In-6D2 and the irrelevant IgM 12A1 (which binds to C. neoformans capsule) was evaluated by incubating 2 μg/mL mAb with 0.23-2×10⁶ whole cells of the human lightly pigmented melanoma line SK-28-MEL (ATCC) which was grown with or without 110 μM L-tyrosine to promote melanin formation. Cell binding of ¹¹¹In-6D2 (FIG. 1A) was higher for the melanoma cells grown with 110 μM L-tyrosine suggesting melanin-specific binding. The binding of ¹¹¹In-6D2 antibody to SK-MEL-28 cells was due to the presence of extracellular melanin in the milieu which is constantly being released as a result of cell turn-over. For MNT1 cells significantly higher binding was observed for lysed cells which is almost certainly due to the release of melanin from the cells and making it accessible to the antibody (FIG. 1B).

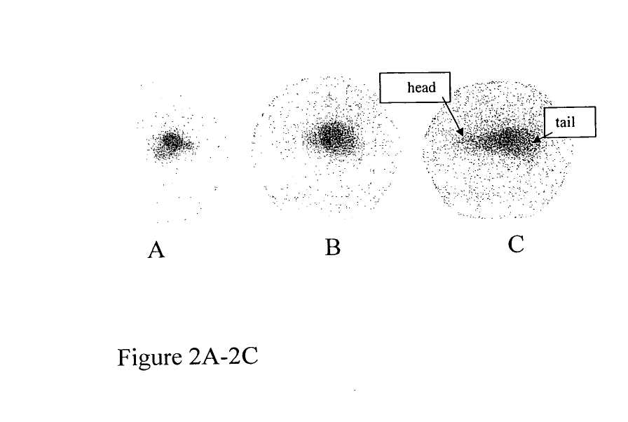

In vivo binding of radiolabeled antibody to SK-28-MEL melanoma cells. In vivo binding of ¹¹¹In-6D2 and control ¹¹¹In-IgM was studied by scintigraphic imaging in nude mice injected IP with 2.8×10⁶ SK-28-MEL cells 24 hours before ¹¹¹In-6D2 (FIG. 2A-2C). For comparison several non-tumor bearing mice were injected IP with ¹¹¹In-6D2 mAb. In mice injected IP with SK-28-MEL cells there was more retention of ¹¹¹In-6D2 in the intraperitoneal cavity (FIG. 2A) compared to irrelevant ¹¹¹In-IgM (FIG. 2B) and control animals with no tumor cells (FIG. 2C).

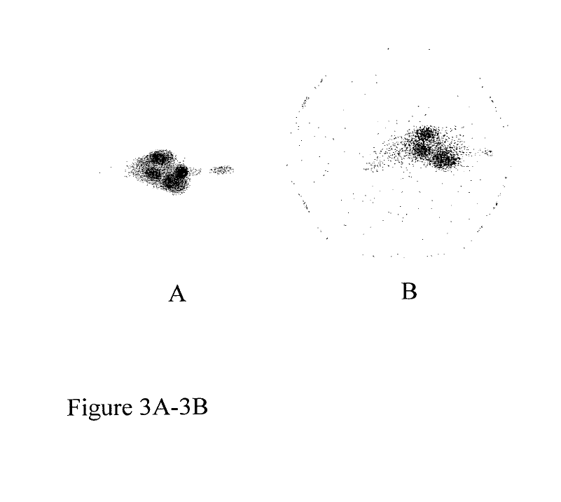

Radioimmunotherapy of melanomas using radiolabeled antibody to melanin. Melanoma-like lesions were created in nude mice using highly pigmented human melanoma cells MNT1 (27). Tumors were induced by injecting 5.5×10⁶ MNT1 cells into the right flank of female nude mice. The tumors reached 0.7-1 cm in diameter 4 weeks after implantation. Several tumor-bearing and control (no tumors) mice were imaged with 0.4 mCi ^{99m}Tc-6D2 (IV injection). Excellent localization in the tumor was achieved at 3 hours (FIG. 3A) and remained high at 24 hours (FIG. 3B). Significant uptake of ^{99m}Tc-6D2 antibody was also observed in kidneys which is, most likely, due to the fact that the complimentarity determining region (CDR) of the antibody carries a positive charge which attracts antibody molecules to the negatively charged sites in the membranes of renal tubular cells (28). Alternatively, damaged radiolabeled antibody molecules may be cleared by the kidney. Blocking the kidneys with positively charged amino acids, such as D-lysine (29, 30), or using better defined preparations of labeled IgM may help in circumventing uptake of antibody by the kidney.

Binding of anti-melanin mAb 6D2 to MNT1 melanoma cells in vitro. To prove that mAb 6D2, which had been developed against fungal melanin, could also bind to tumor-derived melanin and to elucidate the mechanism of mAb 6D2 binding to MNT1 melanoma cells in vitro, the binding of this mAb to MNT1 cells was studied by immunofluorescence. The mAb 6D2 bound only to dead melanoma cells which comprised 3-5% of the total number of cells in culture as measured by the dye exclusion assay. Dead cells apparently released their melanin or had disrupted cell membranes that allowed antibody access to melanin. No binding was observed to viable cells with intact cell membranes. Control mAb 5C11 did not bind to either viable or dead MNT1 cells (results not shown).

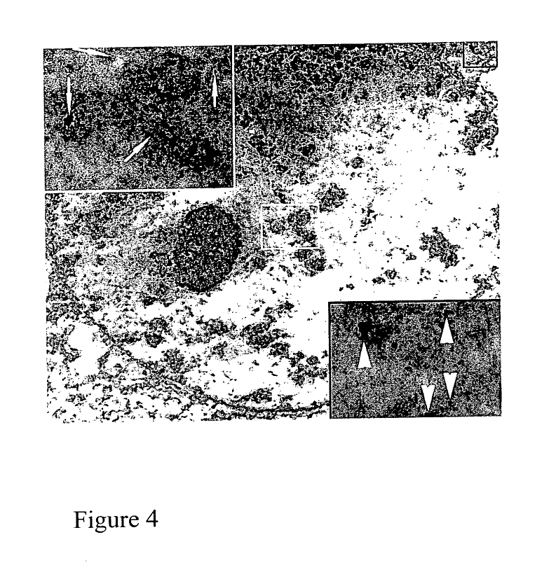

Binding of anti-melanin mAb 6D2 to tumor melanin. Immunogold transmission electron microscopy TEM experiments were performed to establish at the ultrastructural level whether mAb 6D2 could interact with tumor melanin. TEM of the tumor tissue arising from the human melanoma cell line MNT1 implanted into nude mice proved that mAb 6D2 bound to tumor melanin synthesized in vivo. Gold balls were associated with melanin particles inside the cell (FIG. 4, upper insert). By this method cytoplasmic melanin is made accessible to the antibody when the tissue is sectioned. However, the presence of extracellular melanin was almost certainly the result of melanin release from melanoma cells undergoing rapid cell turnover in a fast growing tumor (FIG. 4, lower inset). No association of gold balls with melanin was observed when the tumor tissue was stained with an irrelevant IgM (not shown).

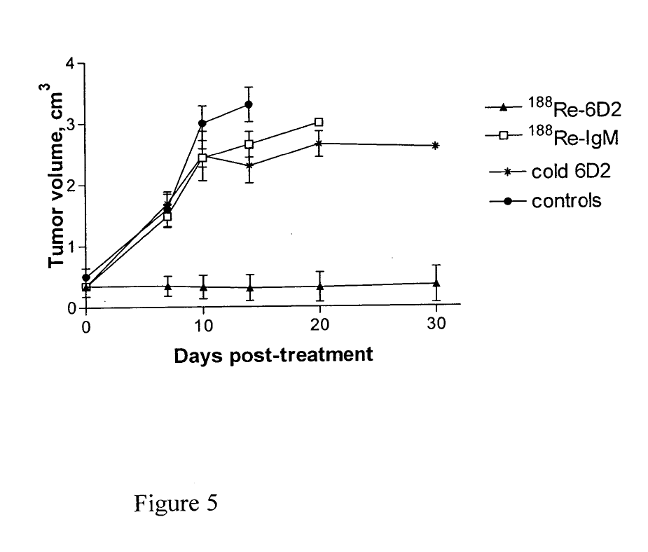

Therapy of MNT1 melanoma in nude mice with ¹⁸⁸Re-6D2. For therapy experiments MNT1 tumor-bearing mice were separated into 4 groups of 7-8 animals and treated IV with: 1.5 mCi ⁸⁸Re-6D2; 1.5 mCi ¹⁸⁸Re-IgM; 100 μg unlabeled 6D2 or left untreated. Growth was completely inhibited in the group treated with ¹⁸⁸Re-6D2 (FIG. 5), and tumor regression occurred in animals with smaller initial tumors. Residual thin (˜1 mm) melanin plaques remained in mice with regressed tumors until they were sacrificed at day 30 after treatment. During the observation period, no deaths occurred in the mice treated with ¹⁸⁸Re-6D2. In contrast, tumors continued to grow aggressively in mice treated with ¹⁸⁸Re-IgM or unlabeled 6D2 and in the untreated mice. By day 20 post-treatment all control mice, except for one in the unlabeled 6D2 group, had died.

Safety of RIT of melanoma with melanin-binding antibodies. Comparative scintigraphic imaging of black and white mice with ¹⁸⁸Re-6D2 mAb. In order to determine if ¹⁸⁸Re-6D2 mAb binds to normal melanocytes, comparative imaging was performed using C57BL/6 black mice and BALB/c white mice. C57BL/6 mice have black hair, black eyes and melanized skin on their tails. Six C57BL/6 and six white BALB/c mice were injected IV with the same activity used in therapy experiments −1.5 mCi ¹⁸⁸Re-6D2. Mice were imaged on a gamma camera 3 and 24 hours post-injection.

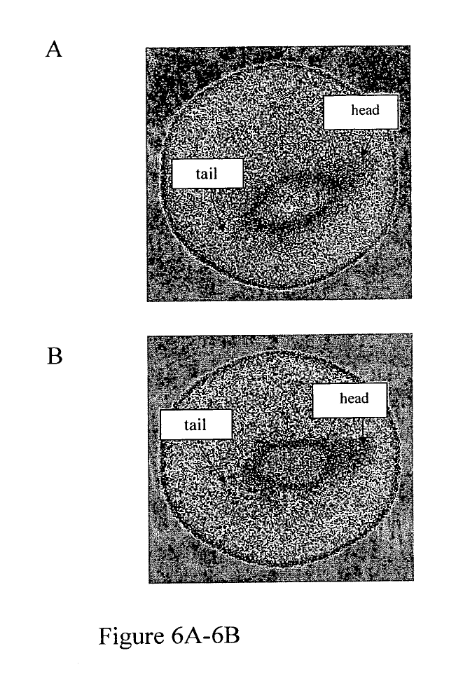

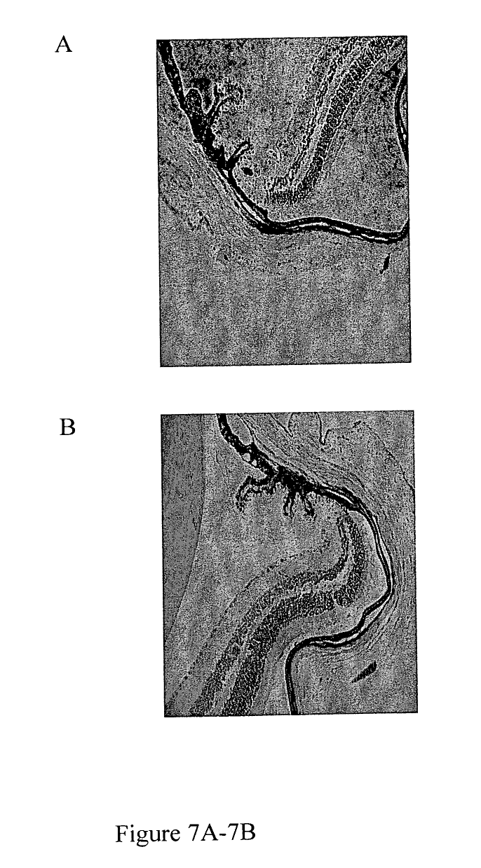

No uptake of ¹⁸⁸Re-6D2 was detected in the hair follicles, eyes, brains and melanized tails of C57BL/6 black mice at 3 hours (FIG. 6A) and at 24 hours (not shown) post-injection in comparison with white BALB/c mice (FIG. 6B). In order to determine if any radiation damage was induced by ¹⁸⁸Re-6D2 mAb in melanin-containing normal tissues, three C57BL/6 black mice imaged with 1.5 mCi ¹⁸⁸Re-6D2 and three control C57BL/6 mice were sacrificed 2 weeks post-imaging, followed by three other imaged and three other control mice at 4 weeks post-imaging. Their eyes and melanized skin from the tails were removed, formalin-fixed, paraffin-embedded, stained with hematoxylin and eosin, and analysed histologically. No radiation damage was detected in the eyes (FIG. 7) and melanized skin (results not shown) of C57BL/6 black mice treated with ¹⁸⁸Re-6D2 mAb.

Melanin-binding peptide synthesis, radiolabeling and serum stability. The availability of Fmoc-HYNIC reagent provided a convenient way of synthesizing melanin-binding and irrelevant control peptides modified with HYNIC ligand at the N-terminus. Radiolabeling of these peptides with ¹⁸⁸Re resulted in 55-65% labeling yields. Purification using SEP-PAK18 chromatographic cartridges achieved 95-99% radiochemical purity. Utilization of D-amino acids as well as introduction of HYNIC moiety into the molecules proved to be useful for serum stability of both decapeptides, as at 5 hours incubation in mouse serum 70-75% of ¹⁸⁸Re activity was still associated with the peptides and not with plasma proteins.

Serum stability of 188-Re-HYNIC-L-4B4 and 188-Re-HYNIC-D-4B4. Both L- and D-4B4 peptide radiolabeled with 188-Re through HYNIC ligand were incubated in mouse serum at 37° C., and aliquots were withdrawn at 0, 0.5, 1, 2, 3, 4 and 5 hours and analyzed on size exclusion HPLC column eluted with PBS, pH 7.2 at 1 mL/min. Peptide and proteins were monitored by UV detector; 1 mL fractions were collected and counted in a dose calibrator. Utilization of D-amino acids proved to be useful for serum stability of peptide, as at 5 hours incubation in mouse serum 70-75% of 188-Re activity was still associated with the D-peptide and not with plasma proteins versus just 25-30% activity associated with L-peptide.

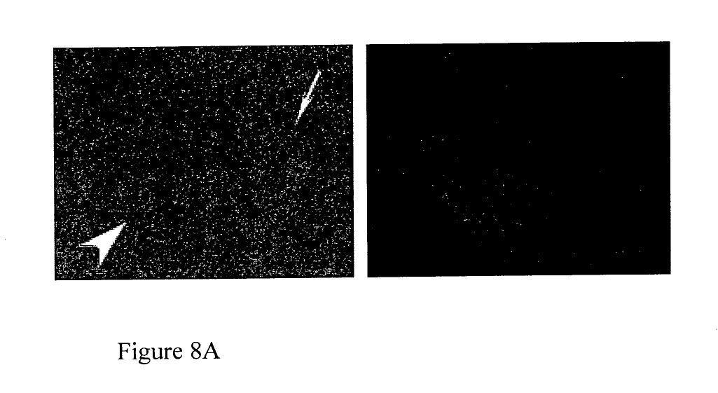

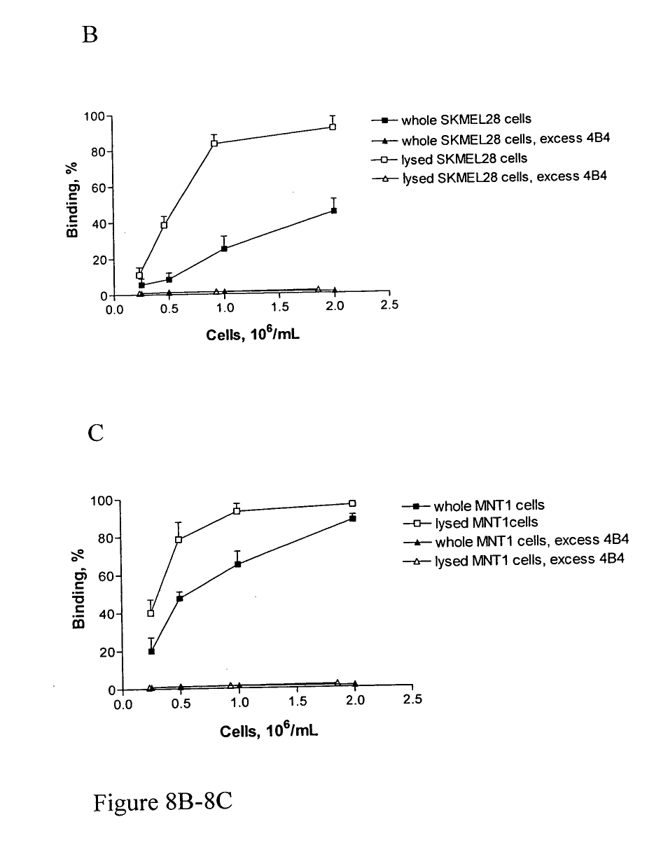

¹⁸⁸Re-HYNIC-4B4 peptide bound only to non-viable melanoma cells in vitro. Peptide binding to melanin in MNT1 cells was studied by immunofluorescence. The biotinylated 4B4 peptide bound only to non-viable melanoma cells (FIG. 8A, arrowhead), which comprised 3-5% of the total cells in culture as measured by the dye exclusion assay. Non-viable cells apparently released their melanin or had permeable cell membranes that allowed access by the peptide to melanin (FIG. 8A left panel). No binding was observed to viable cells with intact cell membranes (FIG. 8A, arrow). Control biotinylated decapeptide PA1 did not bind to either viable or dead MNT1 cells.

¹⁸⁸Re-HYNIC-4B4 readily bound to both human melanoma cells lines employed in this study—slightly pigmented cells SK-28-MEL and highly pigmented cells MNT1 (FIG. 8B-8C). The binding was specific to melanin as pre-incubating the cells with excess of unlabeled HYNIC-4B4 effectively blocked the subsequent binding of ¹⁸⁸Re-HYNIC-4B4. To investigate whether there was a correlation between the amount of extracellular melanin and the peptide binding, binding to whole cells was compared to binding to lysed cells. For both cell lines significantly higher binding to lysed cells was observed.

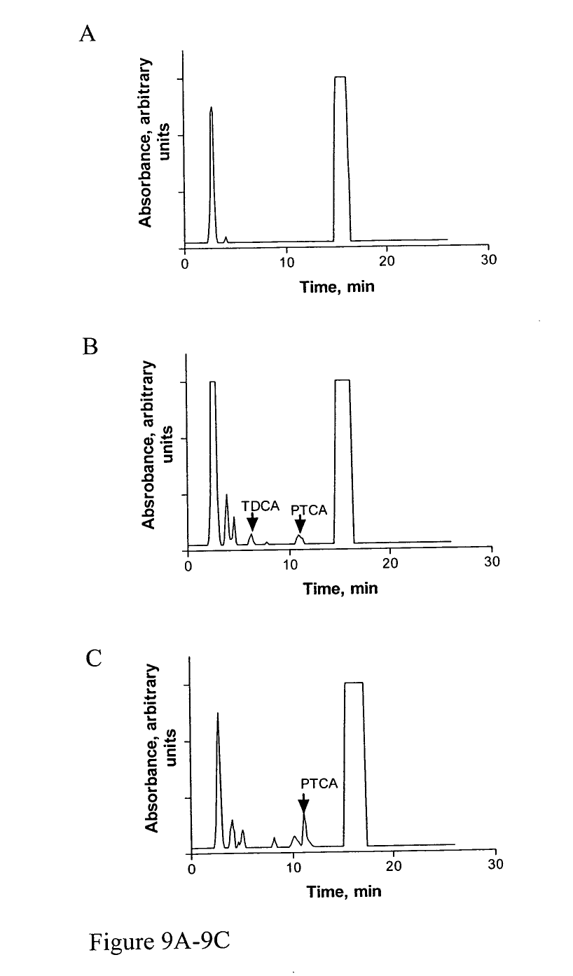

HPLC analysis of melanin from SK-28-MEL and MNT1 melanoma cells. HPLC analysis of the yellowish-brown melanin from SK-MEL-28 cells and the black melanin from MNT1 cells was done to investigate the structural motifs of melanins from different cell lines that may be responsible for the binding of 4B4 peptide. The background solution (consisting of 1.85% Na₂SO₃ and 0.012% KMnO₄ in 0.18 M H₂SO₄) used for oxidation of melanin had peaks that eluted at 2.7 and 15.7 min (FIG. 9A), and the PTCA and TDCA standards eluted at 11 and 6.2 min, respectively (results not shown). Oxidized melanin from MNT1 cells yielded one peak at 11 min that was assigned to PTCA (FIG. 9C). The chromatogram of the oxidized melanin from SK-28-MEL cells had both PTCA and TDCA peaks that were smaller than the PTCA peak in the MNT1 chromatogram, consistent with the lower quantity of melanin in the SK-28-MEL cells (FIG. 9B). The absence of a TDCA peak in the MNT1 chromatogram may be explained by the fact that black coloration of the tumors is caused by the presence of eumelanin, while TDCA is primarily a product of pheomelanin oxidation (66).

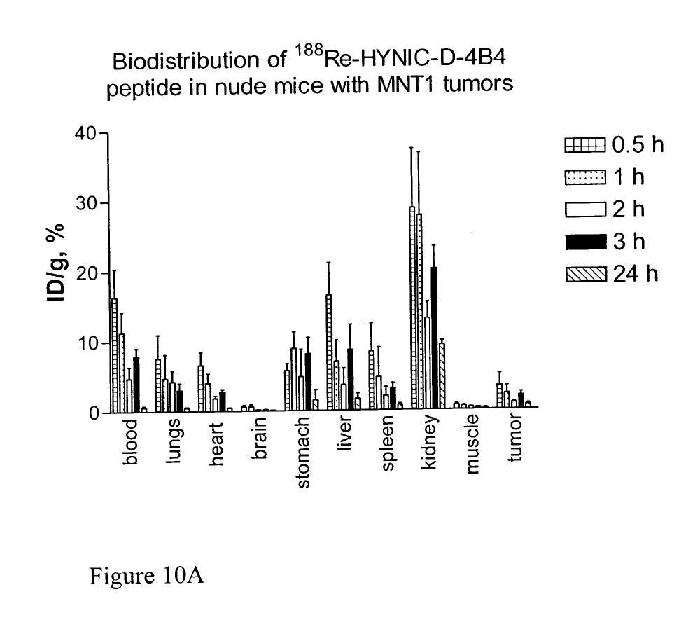

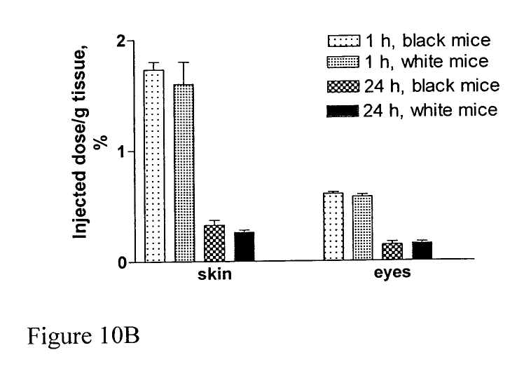

Biodistribution of ¹⁸⁸Re-HYNIC-4B4 in MNT1 tumor-bearing mice. ¹⁸⁸Re-HYNIC-4B4 was cleared rapidly from the blood with only 0.5% ID/g remaining in circulation at 24 h post-injection (FIG. 10A). Interestingly, a transient increase in uptake of practically all major organs was observed at 3 h post-injection which might be explained by the redistribution of activity, possibly, from the intestinal compartment. The kidney uptake was high with ˜30% ID/g at 0.5-1 h post-injection which decreased to 10% ID/g at 24 h, closely resembling the biodistribution pattern of melanin-binding mAb ¹⁸⁸Re-6D2. The tumor uptake of ¹⁸⁸Re-HYNIC-4B4 was highest at the earlier time intervals (˜4.5% ID/g) and decreased to 0.5% ID/g at 24 h. At all times uptake of ¹⁸⁸Re-HYNIC-4B4 in the tumor was 10 times higher than in the surrounding muscle tissue.

No statistically significant difference was observed between uptake of ¹⁸⁸Re-HYNIC-4B4 in the eyes and the skin of black C57BL6 mice in comparison with that in white BALB/c mice (FIG. 10B) which is consistent with the inaccessibility of melanin pigment in healthy melanized tissues to ¹⁸⁸Re-HYNIC-4B4 peptide.

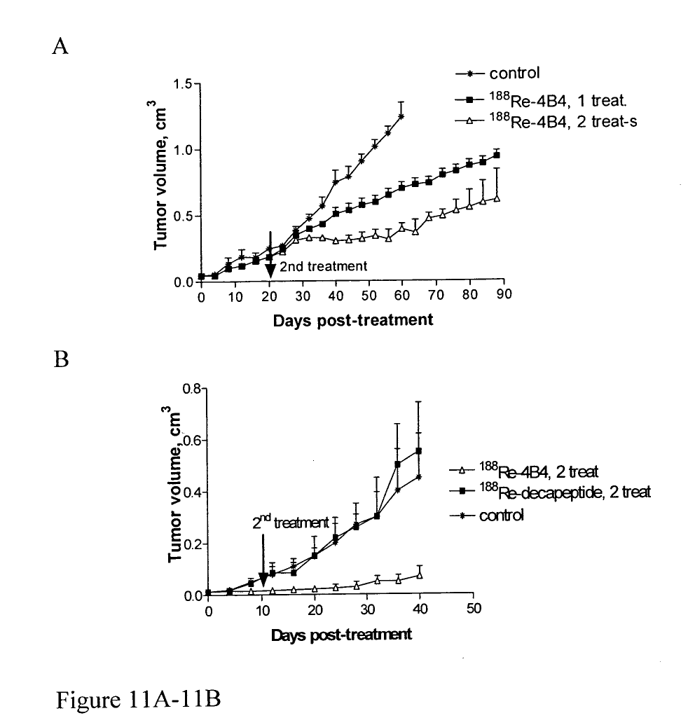

Therapy of MNT1 melanoma in nude mice with ¹⁸⁸Re-HYNIC-4B4 and tumor and kidney dosimetry. In the initial study to examine the effect of radiolabeled melanin-binding peptide on MNT1 tumors, 3 groups of 10 MNT1 tumor-bearing nude mice with 0.5-0.7 cm in diameter were treated IP with: 1) 1.0 mCi ¹⁸⁸Re-HYNIC-4B4; 2) 2×1.0 mCi ¹⁸⁸Re-HYNIC-4B4 20 days apart; or 3) left untreated. The tumors grew in the control group and the last surviving mouse had to be sacrificed on Day 52 because of the size of its tumor. A somewhat slower tumor growth was observed in the group that received one treatment with ¹⁸⁸Re-HYNIC-4B4, and significantly slower growth occurred (P=0.01) in the group treated twice (FIG. 11A).

The 2nd therapy study investigated the influence of tumor size on the therapy results as well as any possible effect on tumor growth from radiation delivered by non-specifically bound decapeptide. Injection of animals bearing 0.3-0.4 cm diameter tumors with 2×1.0 mCi ¹⁸⁸Re-HYNIC-PA1 10 days apart did not have any therapeutic effect on the tumor, while 2×1.0 mCi ¹⁸⁸Re-HYNIC-4B4 administered according to the same regimen completely arrested the growth of the tumors until day 20 post-treatment, with tumors subsequently resuming growth at a significantly slower rate than the larger tumors in the 1st study. These results demonstrate that the tumoricidal effect of ¹⁸⁸Re-HYNIC-4B4 was due to its specific binding to melanin in the tumor and that tumors with smaller diameters were more susceptible to treatment with radiolabeled peptide than larger ones. The dose delivered to the MNT1 tumor by 1 mCi ¹⁸⁸Re-HYNIC-4B4 was calculated to be 300 cGy, while the dose to the kidneys was 900 cGy.

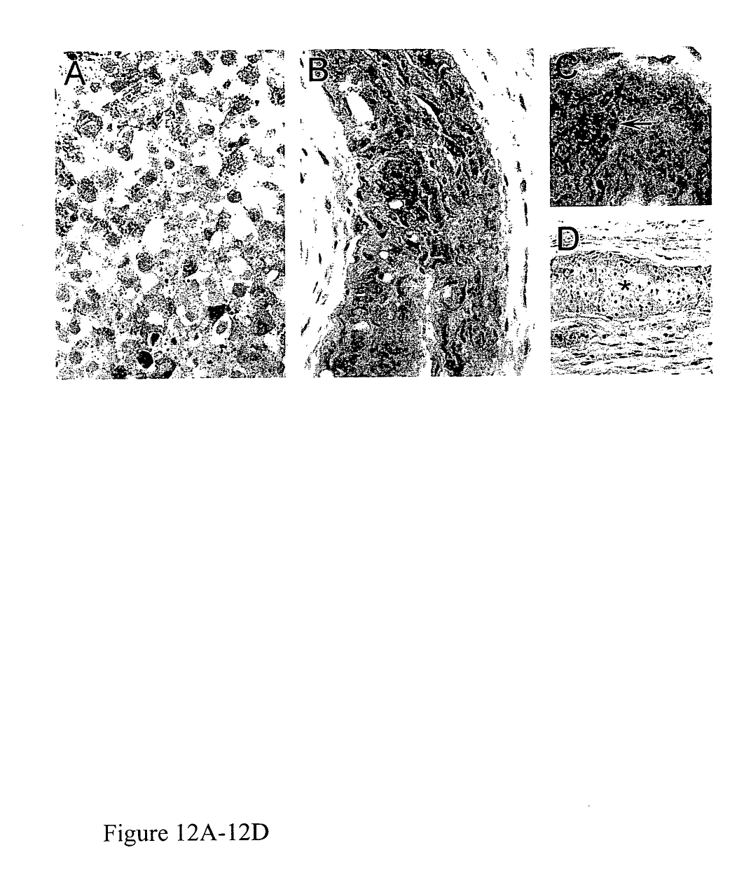

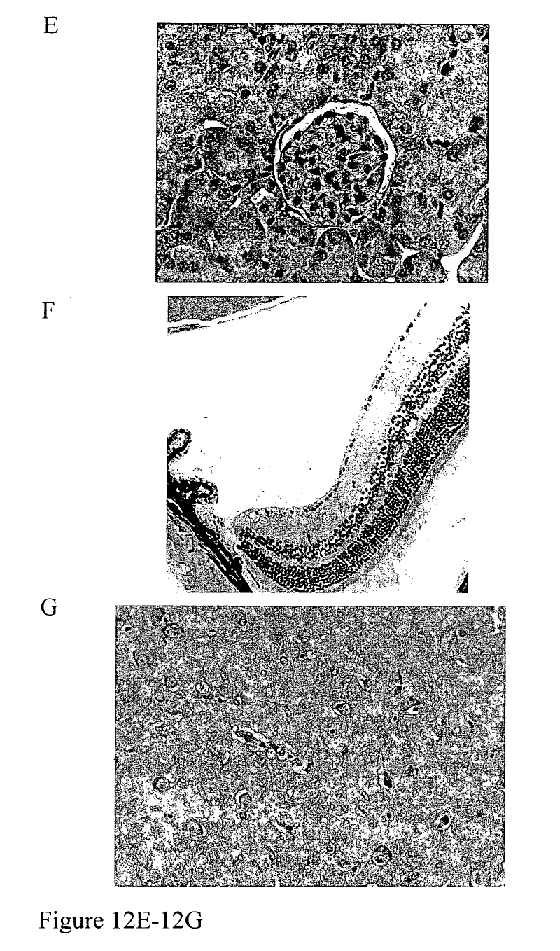

Control mice injected with MNT1 cells all developed gross tumors which were composed of malignant melanoma cells (FIG. 12A). In most mice treated with 2×1 mCi ¹⁸⁸Re-HYNIC-4B4 in the 2nd study no residual malignant melanoma cells were identifiable (FIG. 12B). Only areas of fibrosis with phagocytic histiocytes associated with intra- and extracellular melanin pigment and a rare iron granule were found in the areas where the MNT1 cells had been injected (FIG. 12C,D).

Histological evaluation of ¹⁸⁸Re-HYNIC-4B4 toxicity to kidneys and normal melanized tissues. The kidneys of ¹⁸⁸Re-HYNIC-4B4-treated mice revealed normal glomeruli and tubules without signs of fibrosis, vasculitis, or neoplasm (FIG. 12E). No damage was apparent in the eyes (FIG. 12F) and melanocytes in the skin (data not shown) of C57BL6 mice sacrificed 2 months post-treatment.

Behavioral and histological assessment of brain toxicity of ¹⁸⁸Re-HYNIC-4B4. C57BL/6 mice treated with 2×1 mCi ¹⁸⁸Re-HYNIC-4B4 and control mice were subjected to comprehensive behavioral assessment using SHIRPA protocol 1 week and 2 months post-treatment. Each parameter of animal behavior was scored on a scale assigned in SHIRPA to this particular parameter. Results of the assessment 1 week post-treatment are presented in Table 1. There were no significant differences in the behavior of control and treated mice with the possible exception of the touch escape response, which was more vigorous in ¹⁸⁸Re-HYNIC-4B4-treated mice. Interestingly, treated mice were on average 1.2 g heavier than control mice at the 1 week evaluation. The 2nd behavioral assessment performed 2 months post-treatment showed that the body weight equalized between control and treated groups, and no significant differences in behavioral parameters were observed. At the end of 2 months observation mice were killed and their brains examined for histological evidence of neuronal damage (FIG. 12G). No difference was found between ¹⁸⁸Re-HYNIC-4B4-treated and control mice.

TABLE 1
Behavioral observation profile performed according to SHIRPA protocol
for control and treated with 2 × 1 mCi doses of ¹⁸⁸Re-HYNIC-4B4 C57BL6
mice one week post-treatment. Each parameter of animal behavior was scored
on a scale assigned in SHIRPA to this particular parameter.

| | Control Mice | | | | | | Treated mice | | | | | |
|---|---|---|---|---|---|---|---|---|---|---|---|---|
| Parameter | 1 | 2 | 3 | 4 | 5 | Aver. | 1 | 2 | 3 | 4 | 5 | Aver. |
| Weight | 18.4 | 18 | 17.7 | 18.1 | 18 | 18 | 18.9 | 20 | 19.8 | 18.2 | 19 | 19.24 |
| Body position | 5 | 4 | 5 | 4 | 4 | 4.4 | 5 | 4 | 4 | 5 | 4 | 4.4 |
| Spontaneous Activity | 3 | 2 | 3 | 2 | 2 | 2.4 | 2 | 2 | 2 | 3 | 2 | 2.2 |
| Respiration Rate | 2 | 2 | 2 | 2 | 2 | 2 | 2 | 2 | 2 | 2 | 2 | 2 |
| Tremor | 1 | 0 | 1 | 1 | 1 | 0.8 | 1 | 1 | 1 | 1 | 1 | 1 |
| Transfer Arousal | 5 | 5 | 5 | 4 | 5 | 4.8 | 5 | 4 | 5 | 5 | 5 | 4.8 |
| Locomotor Activity | 5 | 6 | 5 | 3 | 4 | 4.6 | 5 | 5 | 6 | 5 | 4 | 5 |
| Palpebral Closure | 0 | 0 | 0 | 0 | 0 | 0 | 0 | 0 | 0 | 0 | 0 | 0 |
| Pilorection | 0 | 0 | 0 | 0 | 0 | 0 | 0 | 0 | 0 | 0 | 0 | 0 |
| Startle Response | 0 | 1 | 0 | 0 | 2 | 0.6 | 2 | 0 | 1 | 0 | 0 | 0.6 |
| Gait | 0 | 0 | 0 | 0 | 0 | 0 | 0 | 0 | 0 | 0 | 0 | 0 |
| Pelvic Elevation | 2 | 1 | 2 | 2 | 2 | 1.8 | 2 | 2 | 2 | 2 | 2 | 2 |
| Tail Elevation | 1 | 1 | 1 | 1 | 1 | 1 | 1 | 1 | 1 | 1 | 1 | 1 |
| Touch escape | 2 | 1 | 1 | 1 | 2 | 1.4 | 3 | 1 | 3 | 2 | 3 | 2.4 |
| Positional Passivity | 0 | 0 | 0 | 0 | 0 | 0 | 0 | 0 | 0 | 0 | 0 | 0 |
| Trunk Curl | 0 | 1 | 1 | 0 | 1 | 0.6 | 1 | 1 | 1 | 1 | 1 | 1 |
| Limb Grasping | 1 | 1 | 1 | 1 | 1 | 1 | 1 | 1 | 1 | 1 | 1 | 1 |
| Visual Placing | 3 | 3 | 4 | 3 | 4 | 3.4 | 3 | 4 | 3 | 3 | 3 | 3.22 |
| Grip Strength | 2 | 4 | 4 | 3 | 3 | 3.2 | 3 | 2 | 2 | 3 | 4 | 2.8 |
| Body Tone | 1 | 1 | 1 | 1 | 1 | 1 | 1 | 1 | 1 | 1 | 1 | 1 |
| Pinna Reflex | 1 | 1 | 1 | 1 | 1 | 1 | 1 | 1 | 1 | 1 | 1 | 1 |
| Corneal Reflex | 1 | 1 | 1 | 1 | 1 | 1 | 1 | 1 | 1 | 1 | 1 | 1 |
| Toe Pinch | 3 | 3 | 2 | 3 | 3 | 2.8 | 3 | 3 | 3 | 3 | 2 | 2.8 |
| Wire Maneuver | 1 | 0 | 1 | 1 | 0 | 0.6 | 0 | 1 | 0 | 1 | 0 | 0.4 |
| Lacrimation | 0 | 0 | 0 | 0 | 0 | 0 | 0 | 0 | 0 | 0 | 0 | 0 |
| Salivation | 0 | 0 | 0 | 0 | 0 | 0 | 0 | 0 | 0 | 0 | 0 | 0 |
| Provoked Biting | 1 | 0 | 1 | 0 | 0 | 0.4 | 0 | 1 | 0 | 1 | 1 | 0.6 |
| Rigthing Reflex | 0 | 0 | 0 | 0 | 0 | 0 | 0 | 0 | 0 | 0 | 0 | 0 |
| Negative Geotaxis | 0 | 0 | 0 | 0 | 0 | 0 | 0 | 0 | 0 | 0 | 0 | 0 |
| Irritability | 0 | 0 | 0 | 0 | 0 | 0 | 0 | 0 | 0 | 0 | 1 | 0.2 |
| Aggression | 0 | 0 | 0 | 0 | 0 | 0 | 0 | 0 | 0 | 0 | 0 | 0 |
| Vocalization | 0 | 0 | 0 | 0 | 0 | 0 | 0 | 0 | 0 | 0 | 1 | 0.2 |

4. Prospective Examples

Generation of antibodies to cellular components that become accessible as a result of cell death. In addition to using anti-melanin antibodies, one can generate antibodies against proteins and other cellular components, such as histones, mitochondrial proteins, and cytoplasmic proteins, which are expressed only intracellularly in high concentrations and are released by dying cells, using well established hybridoma technology described below.

Generation of antibodies to human melanin. In addition to using the anti-melanin antibody illustrated herein, new mAbs to human melanin can be generated using for example melanin from melanoma cells as described below. Melanoma melanin can be purified as described in (40). Briefly, MNT1 highly pigmented human melanoma cells can be grown in MEM/20% FBS medium as in (27), collected and treated with the sequence of cell wall-lysing enzymes, 4 M guanidine thiocyanate and Proteinase K, and boiled in 6 M HCl. The isolated melanin can be extensively dialyzed against deionized water, dried and stored at −20° C.

IgG antibodies against melanoma melanin can be produced by hybridoma technology. Mice will be immunized with purified melanin. The melanin can be used with or without adjuvant. Freund's complete adjuvant for initial immunization followed by Freund's incomplete adjuvant can be used. CpG (unmethylated cytosine-guanine dinucleotides) has been shown to be a highly effective (enhances T cell help) and safe immunogen (42). CpG will be used according to the manufacturer's recommendations (ImmunoEasy Mouse Adjuvant, Qiagen). Priming with heat-killed MNT1 melanoma cells followed by boosting with the protein can also be used. Although the intraperitoneal approach has been used in the immunizations that identified mAb 6D2 to C. neoformans melanin (23), the base of tail route appears to be a more effective route since the lymph nodes in this region drain directly into the peritoneal lymph nodes that are rich in dendritic cells, which are considered to be the first line of antigen presentation (43). Serum will be obtained prior to immunization and at various times following immunization. Effectiveness of immunization can be determined by incubation of serially diluted serum in 96 well plates coated with melanin that has been blocked for non-specific binding. Following incubation of the serum, the wells will be washed and alkaline phosphatase (AP)-labeled goat anti-mouse (GAM) IgG/M will be applied. The reaction will be developed with p-nitrophenyl phosphate substrate (p-NPP) and measured at an OD of 405 nm. Pre-immune serum will be compared to serum obtained after immunization for each mouse. For responder mice, the isotypes of the mAbs will be characterized using the ELISA with specific immunoglobulin isotypes.

Splenocytes from mice with strong antibody responses to immunization will be fused with non-producing myeloma partners (23). Hybridomas will be generated by a fusion of spleen cells to myeloma cells at a 4:1 ratio in the presence of 50% polyethyleneglycol. The cell mixture will be suspended in a defined complete hypoxanthine-aminopterin-thymidine (HAT) media, with L-glutamine containing 20% heat-inactivated fetal bovine serum, 10% NCTC-109, HAT, and 1% nonessential amino acids for selection of hybridomas, plated in 96-well tissue culture plates, and incubated in a 10% CO₂ incubator at 37° C. Screening of the hybridomas for the presence of mAbs to the melanin antigen will be performed by incubation of supernatants in 96 well plates coated with immunogen then blocked to prevent non-specific binding. The wells will be washed and AP-labeled (GAM) IgG/M will be applied. The reaction will be developed with p-NPP and measured at an OD of 405 nm. The isotypes of the mAbs will be characterized. Large volumes of supernatant will be generated from the selected hybridomas, purified by a column of agarose beads labeled with Ab to the appropriate mouse immunoglobulin (Sigma), and concentrated by centrifugation in an 100,000 NMWL Ultrafree®-15 centrifugal filter device (Millipore).

Generation of anti-melanoma melanin IgG F(ab′)₂ and Fab′ fragments. F(ab′)₂ fragments can be obtained by use of a commercial kit (ImmunoPure, Pierce) as in (44). Briefly, pepsin digestion of IgG at pH 4.2 will be performed, after which the proteolysis will be stopped by centrifugation of the pepsin beads and by adjusting pH to 7 with 5 M sodium acetate. Fab′ fragments can be generated by incubation of F(ab′)₂ fragments with 10 mM dithiothreitol followed by 22 mM iodoacetamide to block the thiol groups. The molecular weight of the obtained fragments can be analyzed by sodium dodecyl sulfate-polyacrylamide gel electrophoresis (SDS-PAGE) and by size-exclusion HPLC. The protein concentration can be determined by the method of Lowry (45).

Derivatization (conjugation) of anti-melanin antibody and antibody fragments with bifunctional chelating agents. In order to radiolabel an antibody with a radiometal, it is necessary to conjugate a bifunctional chelating agent to the antibody prior to radiolabeling. The choice of radioisotopes to perform quality control of radiolabeled antibodies or in vivo imaging studies is ruled by the concept of “matching pairs” of radiopharmaceuticals. This concept calls for the use of diagnostic isotopes (no α- or β-particles emissions) for imaging procedures and quality control with chemistries similar to those of therapeutic isotopes (46). 111-Indium (¹¹¹In) which is readily available commercially is a substitute for therapeutic isotope ²¹³Bi (46) because of their shared similar chemical properties. Consequently, these isotopes can be used for labeling under identical conditions, and their biodistribution properties are very similar. Hence, the immunoreactivity and, further on, biodistribution results obtained with “In are readily applicable to ²¹³Bi.

For radiolabeling with ¹⁸⁸Re, antibodies and F(ab′)₂ and Fab′ fragments will be derivatized with succinimidyl-HYNIC (hydrazynonicotinamide) and purified as in (47). The advantage of employing “indirect” labeling via bifunctional chelating agents such as HYNIC or others over “direct” labeling is that the radiolabeling process is greatly simplified and shortened by using the aliquots of HYNIC-antibody which can be stored frozen for prolonged periods of time. The incorporation of HYNIC into the proteins will be monitored spectrophotometrically at 385 nm (48). The initial HYNIC to protein ratio will be chosen so that the final HYNIC to protein ratio does not exceed ˜1.5 since above this number a partial loss of mAb's immunoreactivity may occur.

For radiolabeling with ²¹³Bi or ¹¹¹In, antibodies and F(ab′)₂ and Fab′ fragments will be conjugated to N-[2-amino-3-(p-isothiocyanatophenyl)propyl]-trans-cyclohexane-1,2-diamine-N,N′,N″,N′″,N″″-pentaacetic acid (CHXA”) as in (49) with average number of chelates per antibody of ˜1.5 as will be determined by the Yttrium-Arsenazo III spectrophotometric method (50).

Synthesis of tumoral melanin-binding peptides modified at N terminus with biotin for immunofluorescence, CHXA″ and HYNIC ligands for radiolabeling. Fungal melanin-binding peptides have been previously identified and sequenced from the phage display library (24). Tumoral melanin-binding peptides will be identified using the same technique and can be synthesized with biotin, CHXA″ and HYNIC ligands at the N terminus. The structures of the peptides will be verified by mass spectrometry and amino acid sequencing.

Immunoreactivity determination. The immunoreactivity of generated F(ab′)₂ and Fab′ fragments, as well as HYNIC- and CHXA″-derivatized 6D2, whole IgG, its fragments and derivatized 4B4 peptide will be determined by melanoma melanin ELISA as described in (40) and confirmed by immunofluorescence as in (51).

5. Discussion

The present application demonstrates that it is possible to radiolabel an anti-melanin antibody with a variety of isotopes without a loss of immunoreactivity. In addition, the application demonstrates that radiolabeled anti-melanin mAb binds to pigmented melanoma cells and that the binding is directly proportional to the degree of melanization of the cells. Hence, the labeled antibody will localize to a melanoma in a subject. Further, animals xenografted with pigmented human melanoma cells were successfully imaged with ^{99m}Tc-6D2 and treated with ¹⁸⁸Re-6D2 mAb and with ¹⁸⁸Re-HYNIC-D-4B4. Hence, administration of radiolabeled anti-melanin antibody translates into a therapeutic effect.

In normal tissues melanin is contained intracellularly in melanosomes. However, since melanoma tumors have rapid cell turnover, there appears to be significant tissue stores of extracellular melanin, which can be targeted by melanin-binding compounds. In contrast to conventional tumor antigens, melanin is insoluble, resistant to degradation, and can be expected to accumulate in targeted tissues. Hence, melanin may be a particularly attractive target because the efficacy of therapy could increase with each subsequent treatment cycle, as cells are killed and more melanin is released into the extracellular space.

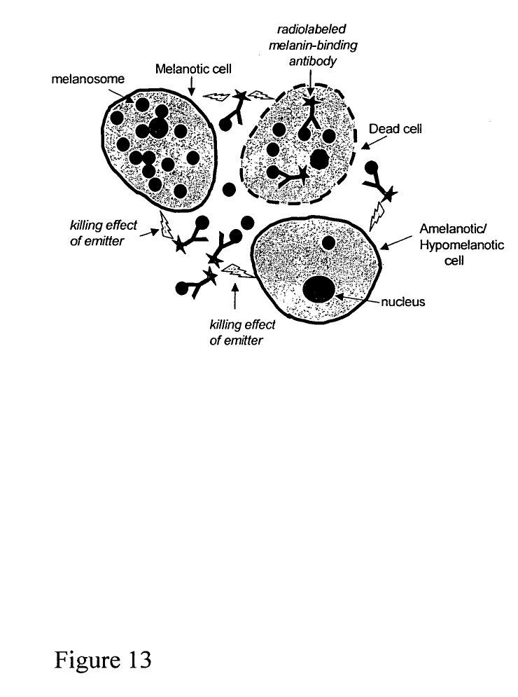

The RIT approach demonstrated herein is useful against melanotic melanomas which constitute the majority of melanomas. It can also be useful against amelanotic melanomas which are generally hypomelanotic (i.e., have small amounts of melanin) rather than truly amelanotic and produce tyrosinase which demonstrates that they can synthesize melanin (61, 62). Targeting melanin with anti-melanin radiolabeled antibodies and peptides should not select for the evolution of a melanotic melanoma into an amelanotic tumor since amelanotic variants in a normal tumor would still be susceptible to killing by the “cross-fire” effect of radiation emanating from the radiolabeled antibody or peptide bound to melanin in the tumor mass (FIG. 13).

In contrast to melanin-binding mAbs, peptides have significantly lower molecular mass, which imply the possibility of delivering radionuclides deeper and more uniformly into the tumor during therapy. They are also less immunogenic and much cheaper than antibodies. The melanin-binding decapeptide 4B4 bound only to non-viable melanoma cells as determined by immunofluorescence. This observation is consistent with the inaccessibility of intracellular melanin in live cells and suggests specificity for tumors with significant cell turnover where melanin would be released extracellularly.

In vitro cell binding showed that radiolabeled ¹⁸⁸Re-HYNIC-4B4 bound to both MNT1 human melanoma cells that were highly pigmented with eumelanin (27), and to the lightly pigmented SK-MEL-28 human melanoma cells which were pigmented with pheomelanin (70). Eumelanin is a heterogeneous dark brown/black pigment, which is believed to consist of 5,6-dihydroxyindole (DHI), 5,6-dihydroxyindole-2-carboxylic acid (DHICA), and pyrrole units in different oxidative states, while pheomelanin, a red/brown pigment, is a complex polymer of subunits similar to those of eumelanin, which is also rich in sulfur (65, 66). HPLC studies of melanin from MNT1 and SK-MEL-28 melanoma cells revealed similar products of oxidative degradation though their ratio was different. Thus, melanin-binding peptides can be used for targeting melanins of various types. This observation is important for metastatic melanoma as both types of melanins are found in melanomas, but eumelanin is the predominant pigment in primary tumors while pheomelanin is associated with progression of the disease (71).

Administration of radiolabeled melanin-binding peptide to MNT1 tumor-bearing mice revealed a therapeutic effect despite a modest uptake of peptide by the tumor (4-5% ID/g). Administration of ¹⁸⁸Re-HYNIC-4B4 significantly slowed tumor growth while the dose delivered to the tumor by the 1 mCi dose was only 300 cGy. This may be explained by deep penetration of the small peptide molecule into the tumor as well as by “cross-fire” irradiation of distant cells allowing relatively homogenous irradiation of the tumor resulting in almost all tumor cells being “hit” by beta-particles. The therapeutic effect of ¹⁸⁸Re-HYNIC-4B4 was due to its specific binding to melanin in the tumor, as treatment of tumor-bearing mice with irrelevant decapeptide labeled with the same activity of ¹⁸⁸Re did not produce any therapeutic gain. Repeated doses of ¹⁸⁸Re-HYNIC-4B4 had a more profound effect on tumor growth that a single dose suggesting the potential effectiveness of multiple administrations of radiolabeled peptide. Treatment of smaller tumors (0.3-0.4 cm in diameter) was more effective in comparison to larger ones (0.5-0.7 cm). The dependence of the efficacy of treatment with radiolabeled peptides on the size of the tumor was reported for [(⁹⁰Y-DOTA)(0),Tyr(3)]octreotide treatment of somatostatin receptor-positive rat pancreatic CA20948 tumors in Lewis rats (72).

No histological evidence of kidney toxicity was found despite a kidney dose of 900 cGy. Clinical trials of radiolabeled peptides suggest that the risk of nephrotoxicity is a function of such characteristics of the peptide molecule as the molecular mass, electric charges and clearance pathways as well as of the chemical and physical characteristics of the applied radionuclide (68). In this regard ¹⁸⁸Re with its relatively short half-life (16.9 h versus 2.8 days for ⁹⁰Y) may have certain advantages over the ⁹⁰Y isotope that has been used so far in most of the patient trials with radiolabeled peptides. Evaluation of potential toxicity to healthy melanized tissues is very important for any melanin-targeting pharmaceutical especially for ones with small molecular mass, which can potentially allow the peptide to penetrate membranes of melanocytes in the eyes, moles, hair follicles etc. In this regard, melanin pre-cursors such as 4-S-cysteaminylphenol (4-S-CAP) that have demonstrated anti-melanoma activity also caused depigmentation of follicular melanocytes in C57BL black mice (73) and iodobenzamides have been found to co-localize with pigmented cells in the eyes and the skin (74). The absence of toxicity of ¹⁸⁸Re-HYNIC-4B4 towards eyes and melanized skin in C57BL6 mice is consistent with the inability of ¹⁸⁸Re-HYNIC-4B4 to penetrate intact cell membrane, probably, due to its high positive charge, which was demonstrated by IF during binding of ¹⁸⁸Re-HYNIC-4B4 to MNT1 cells in vitro. In addition, no toxic effects of ¹⁸⁸Re-HYNIC-4B4 were observed on the cells in substantia nigra in C57BL6 mice. Though it can be argued that neuromelanin is present in mice in very limited amounts (75), the absence of histologically-apparent toxicity of ¹⁸⁸Re-HYNIC-4B4 to neurons and glial cells combined with the overall very low uptake of ¹⁸⁸Re-HYNIC-4B4 in the brain during biodistribution studies and no changes in behavior of treated mice suggest that this approach will have little or no toxicity. Furthermore, no uptake of ¹⁸⁸Re-6D2 antibody was observed in the melanised skin on the tails, in the hair follicles, eyes, or in the brains on scintigraphic images of back C57BL/6 mice, which was confirmed histologically by the absence of radiation damage to these tissues. Thus, antibody or peptide therapy targeting melanin in patients should not harm non-malignant melanized cells such as normal melanocytes or melanin-containing neurons since melanin in healthy tissue can be expected to be intracellular and not accessible to antibody.

In addition to melanomas, other tumors also contain melanin, for example pigmented schwannomas and pigmented neurofibromas (76). The approaches demonstrated herein for treatment of melanoma are also applicable to treatment of other melanin-containing tumors such as pigmented schwannomas and pigmented neurofibromas.

The RIT and radiolabeled peptide approach described herein should also be useful for treating tumors using a radiolabeled antibody that binds to a cellular component released by a dying tumor cell. This techniques is particularly useful for treatment of aggressive, rapidly growing tumors where the cell turnover is much higher than in normal, healthy tissue.

### REFERENCES

1. Rigel D S Malignant melanoma: incidence issues and their effect on diagnosis and therapy in the 1990s. Mayo Clin. Proc., 72: 367-371, 1997.
2. Grin-Jorgensen C M, Rigel D S and Friedman R J The world-wide incidence of malignant melanoma. In: C. M. Balch, A. N. Houghton, G. W. Milton, A. J. Sober, and S. J. Soong (eds.), Cutaneous Melanoma, Ed. 2, pp. 27-39. Philadelphia: J.B. Lippincott Co., 1992.
3. Liu T and Soong S J Epidemiology of malignant melanoma. Surg. Clin. N. Am., 76: 1205-1222, 1996.
4. Sun W, Schuchter L M. Metastatic melanoma. Curr Treat Options Oncol 2: 193-202, 2001.
5. Safa M M and Foon K A Adjuvant immunotherapy for melanoma and colorectal cancers, Semin. Oncol. 28: 68-92, 2001.
6. Kang N V and Yong A. New techniques for imaging metastatic melanoma. Surgery (St. Louis), 16: v-vii, 1998.
7. Larson S M, Carrasquillo J A, Krohn K A Localization of 131-I-labeled p97-specific Fab fragments in human melanoma as a basis for radiotherapy. J. Clin. Invest. 72: 2101-2114, 1983.
8. Goldenberg, D. M. (ed.) Cancer therapy with radiolabeled antibodies. CRC Press, Boca Raton, Fla., 1995.
9. Knox, S J, Meredith, R F. Clinical radioimmunotherapy. Semin. Radiat. Oncol. 10: 73-93, 2000.
10. Satyamoorthy K, Chehab N H, Waterman M J, Lien M C, El-Deiry W S, Herlyn M, Halazonetis T D. Aberrant regulation and function of wild-type p53 in radioresistant melanoma cells. Cell Growth Differ 11: 467-474, 2000.
11. Jenrette J M. Malignant melanoma: the role of radiation therapy revisited. Semin. Oncol. 23: 759-762, 1996.
12. Murtha A D: Review of low-dose-rate radiobiology for clinicians, Semin. Radiation Oncol., 10: 133-138, 2000.
13. Knox S J, Goris M L, Wessels B W. Overview of animal studies comparing radioimmunotherapy with dose equivalent external beam radiation. Radiother. Oncol. 23: 111-117, 1992.
14. Lindmo T, Boven E, Mitchell J B, Morstyn G, Bunn P A Jr. Specific killing of human melanoma cells by ¹²⁵I-labeled 9.2.27 monoclonal antibody. Cancer Res. 45: 5080-5087, 1985.
15. Charlton D E. The survival of monolayers of cells growing in clusters irradiated by 211At appended to the cell surfaces. Radiat Res. 151: 750-753, 1999.
16. Ong G L, Elsamra S E, Goldenberg D M, Mattes M J. Single-cell cytotoxicity with radiolabeled antibodies. Clin Cancer Res 7: 192-201, 2001.
17. Kwok C S, Crivici A, MacGregor W D, Unger M W. Optimization of radioimmunotherapy using human malignant melanoma multicell spheroids as a model. Cancer Res. 49: 3276-3281, 1989.
18. Allen B J, Rizvi S M, Tian Z. Preclinical targeted alpha therapy for subcutaneous melanoma. Melanoma Res 11: 175-182, 2001.
19. Larson S M, Carrasquillo J A, McGuffin R W, Krohn K A, Ferens J M, Hill L D, Beaumier P L, Reynolds J C, Hellstrom K E, Helistrom I. Use of 1-131 labeled, murine Fab against a high molecular weight antigen of human melanoma: preliminary experience. Radiology. 155: 487-492, 1985.
20. Kinnaert E, Morandini R. Simon S, Hill H Z, Ghanem G, Van Houtte P. The degree of pigmentation modulates the radiosensitivity of human melanoma cells. Radiat Res. 154: 497-502, 2000.
21. Hill, H Z The function of melanin or six blind people examine an elephant. Bioessays 14: 49-56, 1992.
22. Rofstad, E K Radiation biology of malignant melanoma. Acta Radiol. Oncol. 25: 1-10, 1986.
23. Rosas A L, Nosanchuk J D, Feldmesser M, Cox G M, McDade H C, Casadevall A. Synthesis of polymerized melanin by Cryptococcus neoformans in infected rodents. Infect Immun. 68: 2845-2853, 2000.
24. Nosanchuk J D, Valadon P, Feldmesser M, Casadevall A. Melanization of Cryptococcus neoformans in murine infection. Mol Cell Biol. 19: 745-750, 1999.
25. Nosanchuk J D, Casadevall A. Cellular charge of Cryptococcus neoformans: contributions from the capsular polysaccharide, melanin, and monoclonal antibody binding. Infect. Immun. 65: 1836-1841, 1997.
26. Blower P J, Clark K, Link E M. Radioiodinated methylene blue for melanoma targeting: chemical character-risation and tumour selectivity of labeled components. Nucl Med Biol 24: 305-310, 1997.
27. Kushimoto T, Basrur V, Valencia J, Matsunaga J, Vieira W D, Ferrans V J, Muller J, Appella E, Hearing V J. A model for melanosome biogenesis based on the purification and analysis of early melanosomes. Proc Natl Acad Sci USA. 98: 10698-10703, 2001.
28. Mogensen C E, Solling K. Studies on renal tubular protein reabsorption: partial and near complete inhibition by certain amino acids. Scand J Clin Lab Invest. 37: 477-486, 1977.
29. Bernard B F, Krenning E P, Breeman W A, Rolleman E J, Bakker W H, Visser T J, Macke H, de Jong M. D-lysine reduction of indium-1111 octreotide and yttrium-90 octreotide renal uptake. J Nucl Med. 38: 1929-1933, 1997.
30. Behr T M, Behe M, Kluge G, Gotthardt M, Schipper M L, Gratz S, Arnold R, Becker W, Goldenberg D M. Nephrotoxicity versus anti-tumour efficacy in radiopeptide therapy: facts and myths about the Scylla and Charybdis. Eur J Nucl Med Mol Imaging. 29: 277-279, 2002.
31. Knapp, F. F. Jr. Rhenium-188—a generator-derived radioisotope for cancer therapy. Cancer Biother Radiopharm. 13: 337-349, 1998.
32. Hoher M., Wohrle J, Wohlfrom M, Hanke H, Voisard R, Osterhues, H H, Kochs M, Reske S N, Hombach V, Kotzerke J. Intracoronary beta-irradiation with a liquid 188-Re-filled balloon: six-month results from a clinical safety and feasibility study. Circulation 101: 2355-2360, 2000.
33. Palmedo H, Guhlke S, Bender H, Sartor J, Schoeneich G, Risse J, Grunwald F, Knapp F. F. Jr, Biersack H J. Dose escalation study with rhenium-188 hydroxyethylidene diphosphonate in prostate cancer patients with osseous metastases. Eur. J. Nucl. Med. 27: 123-130, 2000.
34. McDevitt M R, Barendswaard E, Ma, D, Lai L, Curcio M J, Sgouros G, Ballangrud A M, Yang W H, Finn R D, Pellegrini V. An alpha-particle emitting antibody [213Bi]J591 for radioimmunotherapy of prostate cancer. Cancer Res. 60: 6095-6100, 2000.
35. Kennel S J, and Mirzadeh S. Vascular targeted radioimmunotherapy with 213-Bi—an alpha-particle emitter. Nucl. Med. Biol. 25: 241-246, 1998.
36. Behr T M, Behe M, Stabin M G, Wehrmann E, Apostolidis C, Molinet R, Strutz F, Fayyazi A, Wieland E, Gratz S. High-linear energy transfer (LET) alpha versus low-LET beta emitters in radioimmunotherapy of solid tumors: therapeutic efficacy and dose-limiting toxicity of 213Bi-versus 90Y-labeled CO17-1A Fab′ fragments in human colonic cancer model. Cancer Res. 59: 2635-2643, 1999.
37. Adams G P, Shaller C S, Horak E M, Simmons H H, Dadachova K, Chappell L L, Wu C, Marks J D, Brechbiel M W and Weiner L M. Radio-immunotherapy of established solid tumor xenografts with alpha and beta emitter-conjugated anti-HER2/neu single-chain Fv (scFv) and diabody molecules. Cancer Biother. Radiopharm. 15: 402a. (Abstr.), 2000.
38. Kolbert K S, Hamacher K A, Jurcic J G, Scheinberg D A, Larson S M, Sgouros G. Parametric images of antibody pharmacokinetics in 213Bi-HuM195 therapy of leukimia. J. Nucl. Med. 42: 27-32, 2001.
39. Sgouros G, Ballangrud A M, Jurcic J G, McDevitt M R, Humm J L, Erdi Y E, Mehta B M, Finn R D, Larson S M and Scheinberg D A. Pharmacokinetics and dosimetry of an alpha-particle emitter labeled antibody: 213Bi-HuM195 (anti-CD33) in patients with leukemia. J. Nucl. Med. 40: 1935-1946. 1999.
40. Rosas A L, Nosanchuk J D, Gomez B L, Edens W A, Henson J M, Casadevall A. Isolation and serological analyses of fungal melanins. J Immunol Methods 244: 69-80, 2000.
41. Krieg A M Immune effects and mechanisms of action of CpG motifs. Vaccine 19: 618-622, 2000.
42. Weeratna R D, McCluskie M J, Xu Y, and Davis H L CpG DNA induces stronger immune responses with less toxicity than other adjuvants. Vaccine 18: 1755-1762, 2000.
43. Lelouard, H., E. Gatti, F. Cappello, O. Gresser, V. Camosseto, and P. Pierre. Transient aggregation of ubiquitinated proteins during dendritic cell maturation. Nature 517: 177-182, 2002.
44. Lendvai N, Casadevall A, Liang Z, Goldman D L, Mukherjee J, Zuckier L. Effect of immune mechanisms on the pharmacokinetics and organ distribution of cryptococcal polysaccharide. J Infect Dis. 177: 1647-1659, 1998.
45. Lowry O H, Rosebrough N J, Farr A L, and Randall R J. Protein measurement with the Folin phenol reagent. J. Biol. Chem. 193: 265-275, 1951.
46. Mirzadeh S, Brechbiel M W, Atcher R W, Gansow O A. Radiometal labeling of immunoproteins: covalent linkage of 2-(4-isothiocyanatobenzyl)diethylenetriamine-pentaacetic acid ligands to immunoglobulin. Bioconjug. Chem. 1: 59-65, 1990.
47. Blankenberg F G, Katsikis P D, Tait J F, Davis R E, Naumovski L, Ohtsuki K, Kopiwoda S, Abrams M J, Strauss H W. Imaging of apoptosis (programmed cell death) with 99mTc annexin V. J Nucl Med. 40: 184-191, 1999.
48. King T P, Zhao S W, Lam T Preparation of protein conjugates via intermolecular hydrazone linkage. Biochem. 25: 5774-5779, 1986.
49. Dadachova E, Mirzadeh S, Smith S V, Knapp F F, and Hetherington E L Radiolabelling antibodies with 166-Holmium. Appl. Rad. Isotop. 48: 477-481, 1997.
50. Pippin C G, Parker T A, McMurry T J, and Brechbiel M W. Spectrophotometric method for the determination of a bifunctional DTPA ligand in DTPA-monoclonal antibody conjugates. Bioconjug. Chem. 3: 342-345, 1992.
51. Nosanchuk J D, Rosas A L, and Casadevall A. The antibody response to fungal melanin in mice, J. Immunol. 1998, 160: 6026-6031.
52. Boll R A, Mirzadeh S, and Kennel S J. Optimizations of radiolabeling of immunoproteins with 213-Bi. Radiochim. Acta 79: 145-149, 1997.
53. Abrams M J, Juweid M, tenKate C I, Schwartz D A, Hauser M M, Gaul F E, Fuccello A J, Rubin R H, Strauss H W, Fischman A J. Technetium-99m-human polyclonal IgG radiolabeled via the hydrazino nicotinamide derivative for imaging focal sites of infection in rats. J. Nucl. Med. 31: 2022-2028, 1990.
54. Dadachova E, Mirzadeh S. The role of tin in the direct labelling of proteins with Rhenium-188. Nucl. Med. Biol. 24: 605-608, 1997.
55. Milenic, D. E. 2000. Radioimmunotherapy: designer molecules to potentiate effective therapy. Semin. Radiat. Oncol. 10: 139-155.
56. Buchsbaum, D. J. 2000. Experimental radioimmunotherapy. Semin. Radiat. Oncol. 10: 156-167.
57. Saha G B Fundamentals of Nuclear Pharmacy, Springer, 1997, New York, pp. 139-143
58. Paganelli G., Zoboli S., Cremonesi M. et al Receptor-mediated radionuclide therapy with 90-Y-DOTA-D-Phe-Tyr³-Octreotide: Preliminary report in cancer patients. Cancer Biother. Radiopharm. 14: 477-483, 1999.
59. Early P. J. and Sodee D. B. Principles and Practice of Nuclear Medicine, Mosby, 1995.
60. Abbas A K, Lichtman A H, Pober J S. Cellular and Molecular Immunology, 4^{th} edition, W.B. Saunders Co., Philadelphia, 2000.
61. Busam K J, Hester K, Charles C, Sachs D L, Antonescu C R, Gonzalez S, Halpern A C. Detection of clinically amelanotic malignant melanoma and assessment of its margins by in vivo confocal scanning laser microscopy. Arch Dermatol 2001 July; 137 (7):923-9.
62. Cohen-Solal K A, Crespo-Carbone S M, Namkoong J, Mackason K R, Roberts K G, Reuhl K R, Chen S. Progressive appearance of pigmentation in amelanotic melanoma lesions. Pigment Cell Res 2002 August; 15 (4):282-9.
63. Virgolini I, Traub T, Novotny C, Leimer M, Fuger B, Li S R, Patri P, Pangerl T, Angelberger P, Raderer M, Andreae F, Kurtaran A, Dudczak R. New trends in peptide receptor radioligands. Q J Nucl Med. 2001 45 (2): 153-159.
64. Westlin J-E, Janson E T, Ahlström H, Nilsson S, Öhrvall U, and Öberg K. Scintigraphy using a 111-In-labeled somatostatin analogue for localization of neuroendocrine tumors. Antibody, Immunoconjugates and Radiopharmaceuticals 1992; 5:367-84.
65. Ito S, and Fujita K. Microanalysis of eumelanin and pheomelanin in hair and melanomas by chemical degradation and liquid chromatography. Anal. Biochem. 1985; 144: 527-36.
66. Wakamatsu K, and Ito S. Advanced Chemical Methods in Melanin Determination. Pigment. Cell Res. 2002; 15:174-83.
67. Hui T E, Fisher D R, Kuhn J A et al A mouse model for calculating cross-organ beta doses from Yttrium-90-labeled immunoconjugates. Cancer 1994: 73 (suppl): 951-7.
68. Lambert B, Cybulla M, Weiner S M, et al. Renal toxicity after radionuclide therapy. Radiat Res 2004; 161:607-11.
69. Rogers D C, Fisher E M, Brown S D, Peters J, Hunter A J, Martin J E Behavioral and functional analysis of mouse phenotype: SHIRPA, a proposed protocol for comprehensive phenotype assessment. Mamm Genome 1997; 8:711-13.
70. Rice L, Wainwright M, and Phoenix D A. Phenothiazine photosensitizers: III. Activity of methylene blue derivatives against pigmented melanoma cell lines. J. Chemother 2000; 12: 94-104.
71. Hearing V. The melanosome: the perfect model for cellular responses to the environment. Pigment Cell Res 2000; 13 (Suppl 8):23-34.
72. de Jong M, Breeman W A, Bernard B F, et al Tumor response after [(90)Y-DOTA(0),Tyr(3)]octreotide radionuclide therapy in a transplantable rat tumor model is dependent on tumor size. J Nucl Med 2001; 42:1841-6.
73. Yukitake J, Otake H, Inoue S, Wakamatsu K, Ito S. Comparison of in vivo anti-melanoma effect of enantiomeric alpha-methyl- and alpha-ethyl-4-S-cysteaminylphenol. Melanoma Res. 2004; 14:115-20.
74. Chehade F, De Labriolle-Vaylet C, Michelot J et al Distribution of 1-BZA (N-2-diethylaminoethyl-4-iodobenzamide) in grafted melanoma and normal skin: a study by secondary ion mass spectroscopy (Noisy-le-grand). Cell. Mol. Biol. 2001; 47:529-34.
75. Lindquist N G, Larsson B S, Lyden-Sokolowski A. Autoradiography of [14C]paraquat or [14C]diquat in frogs and mice: accumulation in neuromelanin. Neurosci Lett. 1988; 93:1-6.
76. Boyle J L, Haupt H M, Stern J B, Multhaupt H A. Tyrosinase expression in malignant melanoma, desmoplastic melanoma, and peripheral nerve tumors. Arch Pathol Lab Med. 2002 July; 126 (7):816-22.

1-4. (canceled)

5. A method for treating a melanin-containing tumor in a subject which comprises administering to the subject an amount of a radiolabeled anti-melanin peptide effective to treat the tumor.

6. (canceled)

7. The method of claim 5, wherein the peptide is labeled with an alpha-emitting radioisotope.

8. The method of claim 7, wherein the alpha-emitting radioisotope is 213-Bismuth.

9. The method of claim 5, wherein the peptide is labeled with a beta-emitting radioisotope.

10. The method of claim 9, wherein the beta-emitting radioisotope is 188-Rhenium.

11. The method of claim 5, wherein the peptide is labeled with a radioisotope selected from the group consisting of a positron emitter and an admixture of any of an alpha emitter, a beta emitter, and a positron emitter.

12-15. (canceled)

16. The method of claim 5, wherein the dose of the radioisotope is between 1-1000 mCi.

17-20. (canceled)

21. The method of claim 5, wherein the peptide is positively charged.

22. The method of claim 5, wherein the peptide is a decapeptide.

23. The method of claim 5, wherein the peptide comprises the amino acid sequence YERKFWHGRH (SEQ ID NO:1).

24. The method of claim 5, wherein the peptide is selected from the group consisting of peptides having the amino acid sequence LHKLVRHGRW (SEQ ID NO:2), YLRRHTHVFW (SEQ ID NO:3), KKHSHYWVRY (SEQ ID NO:4), EFGTRHMRHR (SEQ ID NO:5), YRHHAHGGRG (SEQ ID NO:6), RKKWHGWTRW (SEQ ID NO:7), PKWRHGYTRF (SEQ ID NO:8), RHGTVKHARH (SEQ ID NO:9), RRHWHPPVQI (SEQ ID NO:10), EAYKRRWHWP (SEQ ID NO:11), RWPKRHLSGH (SEQ ID NO:12), SRVPFRHYHH (SEQ ID NO:13), RRPEHTKARW (SEQ ID NO:14), WRAFLPRWHA (SEQ ID NO:15), WNRGWRWWMG (SEQ ID NO:16), GFFWKWRIGR (SEQ ID NO:17), and HIRWKGHISW (SEQ ID NO:18).

25. The method of claim 5, wherein the peptide comprises the amino acid sequence X₁-X₂-X₃-X₄-H (SEQ ID NO:19), where X₁ and X₂ are positively charged amino acids, and X₃ and X₄ are positively charged amino acids and/or aromatic amino acids.

26. The method of claim 5, which further comprises administering to the subject anti-melanin antibodies and/or peptides radiolabeled with a plurality of different radioisotopes.

27. The method of claim 26, wherein the radioisotopes are isotopes of a plurality of different elements.

28. The method of claim 26, wherein at least one radioisotope is a long range emitter and at least one radioisotope is a short range emitter.

29. The method of claim 28, wherein the long-range emitter is a beta emitter and the short range emitter is an alpha emitter.

30. The method of claim 29, wherein the beta emitter is 188-Rhenium and the alpha emitter is 213-Bismuth.

31. The method of claim 5, wherein uptake radiolabeled anti-melanin peptide in the tumor is at least 10 times greater than in surrounding muscle.

32. The method of claim 5, wherein the radiolabeled anti-melanin peptide is not taken up by non-cancerous melanin-containing tissue.

33. The method of claim 32, wherein the non-cancerous melanin-containing tissue is hair, eyes, skin, brain, spinal cord, and/or peripheral neurons.

34. The method of claim 5, which comprises multiple administrations of the radiolabeled peptide to the subject.

35-37. (canceled)

38. The method of claim 5, wherein the melanin-containing tumor is a melanoma, a pigmented schwannoma, or a pigmented neurofibroma.

39. The method of claim 5, wherein the peptide is comprised of one or more D-amino acid residues.

40. The method of claim 39, wherein the peptide is comprised of all D-amino acid residues.

41. The method of claim 5, wherein the melanin-containing tumor is a melanoma and wherein the radiolabeled anti-melanin peptide comprises the amino acid sequence YERKFWHGRH (SEQ ID NO:1).

42. The method of claim 41, wherein the peptide is labeled with 188-Rhenium.
